(19) United States
(12) Patent Application Publication
(10) Pub. No.: US 2005/0176796 A1
(43) Pub. Date: Aug. 11, 2005

(54) Tricyclic pyrazole derivatives, process for their preparation and their use as antitumor agents

(76) Inventors: Roberto D'Alessio, Cinisello Balsamo (IT); Alberto Bargiotti, Milano (IT); Maria Gabriella Brasca, Cusago (IT); Antonella Ermoli, Buccinasco (IT); Paolo Pevarello, Pavia (IT); Marcellino Tibolla, Senago (IT)

Correspondence Address: Peter I Bernstein;Scully Scott Murphy & Presser, Suite 300, 400 Garden City Plaza, Garden City, NY, 11530, US

(21) Appl. No.: 10/505,200

(22) PCT Filed: Feb. 18, 2003
(86) PCT No.: PCT/EP03/01594

Related U.S. Application Data
(60) Provisional application No. 60/357,918, filed on Feb. 19, 2002.

(51) Int. Cl. A61K 31/4188; A61K 31/4162

(52) U.S. Cl. 514/393; 514/406

(57) ABSTRACT

Compounds which are tricyclic pyrazole derivatives and analogues thereof, as set forth in the specification, or pharmaceutically acceptable salts thereof, together with pharmaceutical compositions comprising them are disclosed; these compounds or compositions are useful in the treatment of diseases caused by and/or associated with an altered protein kinase activity such as cancer, cell proliferative disorders, Alzheimer's disease, viral infections, auto-immune diseases and neurodegenerative disorders.

### BACKGROUND OF THE INVENTION

1. Field of the Invention

The present invention relates to tricyclic pyrazole derivatives active as kinase inhibitors and, more in particular, it relates to tricyclic pyrazoles and analogues tricyclic heterocyclic derivatives, to a process for their preparation, to pharmaceutical compositions comprising them and to their use as therapeutic agents, particularly in the treatment of diseases linked to disregulated protein kinases.

2. Discussion of Background

The malfunctioning of protein kinases (PKs) is the hallmark of numerous diseases. A large share of the oncogenes and proto-oncogenes involved in human cancers code for PKs. The enhanced activities of PKs are also implicated in many non-malignant diseases, such as benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis. PKs are also implicated in inflammatory conditions and in the multiplication of viruses and parasites. PKs may also play a major role in the pathogenesis and development of neurodegenerative disorders.

For a general reference to PKs malfunctioning or disregulation see, for instance, Current Opinion in Chemical Biology 1999, 3, 459-465.

### SUMMARY OF THE INVENTION

It is an object of the invention to provide compounds which are useful in therapy as agents against a host of diseases caused by and/or associated to a disregulated protein kinase activity.

It is another object to provide compounds which are endowed with multiple protein kinase inhibiting activity.

The present inventors have now discovered that the compounds of the invention, hereinafter shortly referred to as tricyclic pyrazole derivatives, are endowed with multiple protein kinase inhibiting activity and are thus useful in therapy in the treatment of diseases associated with disregulated protein kinases.

More specifically, the compounds of this invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma.

Due to the key role of PKs in the regulation of cellular proliferation, these compounds are also useful in the treatment of a variety of cell proliferative disorders such as, for instance, benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis. The compounds of the invention can be useful in the treatment of Alzheimer's disease, as suggested by the fact that cdk5 is involved in the phosphorylation of tau protein (J. Biochem., 117, 741-749, 1995).

The compounds of the invention are also useful in the treatment and prevention of radiotherapy-induced or chemotherapy-induced alopecia.

The compounds of this invention, as modulators of apoptosis, may also be useful in the treatment of cancer, viral infections, prevention of AIDS development in HIV-infected individuals, autoimmune diseases and neurodegenerative disorders.

The compounds of this invention may be useful in inhibiting tumor angiogenesis and metastasis, as well as in the treatment of organ transplant rejection and host versus graft diseases.

The compounds of the invention are useful as cyclin dependent kinase (cdk) inhibitors and also as inhibitors of other protein kinases such as, for instance, protein kinase C in different isoforms, Met, PAK-4, PAK-5, ZC-1, STLK-2, DDR-2, Aurora 1, Aurora 2, Bub-1, PLK, Chk1, Chk2, HER2, raf1, MEK1, MAPK, EGF-R, PDGF-R, FGF-R, IGF-R, VEGF-R, PI3K, weel kinase, Src, Abl, Akt, ILK, MK-2, IKK-2, Cdc7, Nek, and thus be effective in the treatment of diseases associated with other protein kinases.

### DETAILED DESCRIPTION OF THE INVENTION

Several pyrazoles and analogues thereof are known in the art, for instance as synthetic intermediates or even as therapeutic agents.

As an example, carboxamido-pyrazoles possessing cdk inhibitory activity have been described in U.S. Pat. No. 6,218,418 to Pevarello et al.

Pyrazoles have been described for use in the treatment of inflammation. U.S. Pat. No. 5,134,142 to Matsuo et al describes 1,5-diaryl pyrazoles, and specifically, 1-(4-fluorophenyl)-5-[4-(methylsulfonyl)phenyl]-3-trifluoromethylpyrazole, as having anti-inflammatory activity.

U.S. Pat. No. 4,734,430 discloses benzo- and cycloheptadipyrazoles as bronchodilators; U.S. Pat. No. 3,940,418 describes tricyclic 4,5-dihydrobenz[g]indazoles as anti-inflammatory agents. In addition, R. Hamilton [J. Heterocyclic Chem., 13, 545 (1976)] describes tricyclic 4,5-dihydrobenz[g]indazoles as anti-inflammatory agents. U.S. Pat. No. 5,134,155 describes fused tricyclic pyrazoles having a saturated ring bridging the pyrazole and a phenyl radical as HMG-CoA reductase inhibitors. European publication EP 477,049, published Mar. 25, 1992, describes [4,5-dihydro-1-phenyl-1H-benz[g]indazol-3-yl]amides as having antipsychotic activity. European publication EP 347,773, published Dec. 27, 1989, describes [4,5-dihydro-1-phenyl-1H-benz[g]indazol-3-yl]propanamides as immunostimulants. M. Hashem et al [J. Med. Chem., 19, 229 (1976)] describes fused tricyclic pyrazoles, having a saturated ring bridging the pyrazole and a phenyl radical, as antibiotics.

Certain substituted pyrazolyl-benzenesulfonamides have been described in the literature as synthetic intermediates. Specifically, 4-[5-(4-chlorophenyl)-3-phenyl-1H-pyrazol-1-yl]benzenesulfonamide has been prepared from a pyrazoline compound as an intermediate for compounds having hypoglycemic activity [R. Soliman et al, J. Pharm. Sci., 76, 626 (1987)]. 4-[5-[2-(4-Bromophenyl)-2H-1,2,3-triazol-4-yl]-3-methyl-1H-pyrazol-1-yl]benzenesulfonamide has been prepared from a pyrazoline compound and described as potentially having hypoglycemic activity [H. Mokhtar, Pak. J. Sci. Ind. Res., 31, 762 (1988)]. Similarly, 4-[4-bromo-5-[2-(4-chlorophenyl)-2H-1,2,3-triazol-4-yl]-3-methyl-1H-pyrazol-1-yl]benzenesulfonamide has been prepared [H. Mokhtar et al, Pak. J. Sci. Ind. Res., 34, 9 (1991)].

The phytotoxicity of pyrazole derivatives is described [M. Cocco et al, Il. Farmaco-Ed. Sci., 40, 272 (1985)], specifically for 1-[4-(aminosulfonyl)phenyl]-5-phenyl-1H-pyrazole-3,4-dicarboxylic acid.

The use of styryl pyrazole esters for antidiabetes drugs is described [H. Mokhtar et al, Pharmazie, 33, 649-651 (1978)]. The use of styryl pyrazole carboxylic acids for antidiabetes drugs is described [R. Soliman et al, Pharmazie, 33, 184-5 (1978)]. The use of 4-[3,4,5-trisubstituted-pyrazol-1-yl]benzenesulfonamides as intermediates for sulfonylurea anti-diabetes agents is described, and specifically, 1-[4-(aminosulfonyl)phenyl]-3-methyl-5-phenyl-1H-pyrazole-4-carboxylic acid [R. Soliman et al, J. Pharm. Sci., 72, 1004 (1983)]. A series of 4-[3-substituted methyl-5-phenyl-1H-pyrazol-1-yl]benzenesulfonamides has been prepared as intermediates for anti-diabetes agents, and more specifically, 4-[3-methyl-5-phenyl-1H-pyrazol-1-yl]benzenesulfonaimde [H. Feid-Allah, Pharmazie, 36, 754 (1981)]. In addition, 1-(4-[aminosulfonyl]phenyl)-5-phenylpyrazole-3-carboxylic acid has been prepared from the above described 4-[3-methyl-5-phenyl-1H-pyrazol-1-yl]benzenesulfonamide compound [R. Soliman et al, J. Pharm. Sci., 70, 602 (1981)].

WO 00/27822 discloses tricyclic pyrazole derivatives, WO 00/59901 discloses dihydroindeno pyrazoles, WO 95/15315 discloses diphenyl pyrazole compounds, WO 95/15317 discloses triphenyl pyrazole compounds, WO 95/15318 discloses tri-substituted pyrazole compounds, and WO 96/09293 discloses benz[g]indazolyl derivatives.

WO 95/15316 discloses substituted pyrazolyl benzenesulfamide derivatives.

Accordingly, the present invention provides a method for treating diseases caused by and/or associated with an altered protein kinase activity, by administering to a mammal in need thereof an effective amount of a compound represented by formula (I) 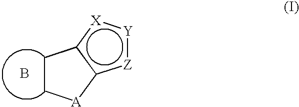
wherein
X, Y and Z, being part of an aromatic ring are selected, each independently, from the group consisting of N, NR₁, S, O and CR₁;
R₁ is selected from the group consisting of hydrido, lower alkyl, perfluorinated lower alkyl, heterocyclyl, CN, CO₂R′, COCF₃, COR′, CONR′R″, NR′R″, C(═NR′)NR′R″, CONHNH₂, CONHOR′, NHCOR′, CH₂NH₂, and CH₂NHCOR′; or R₁ may form, when part of Z or Y, a 5 to 7 membered ring together with the remaining of Y or Z, as per the formulae below 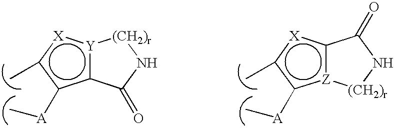
R′ and R″ are selected, each independently, from the group consisting of hydrido, hydroxy, alkyl, hydroxyalkyl, alkenyl, alkynyl, aryl, arylalkyl, heterocyclyl or heterocyclyl-alkyl;
B is an aromatic 5 or 6 membered ring having from 0 to 3 heteroatoms selected from S, O and N;
A is selected from the group consisting of (CH₂)ₘ—, —(CH₂)ₙ—CH═CH—(CH₂)ₙ— and —(CR_{z}R_{y})ₚ—;
R_{z} and R_{y} are selected, each independently, from hydrido or lower alkyl;
each of the X,Y,Z and B rings being optionally further substituted by one or more -L-R₂ groups, wherein L represents, each independently, a single bond, an alkylidene group or a divalent group selected from NH, NHCO, CONH, NHCONH, SO₂NH and NHSO₂;
R₂ is, each independently, hydrido, alkyl, 5 to 12 membered mono- or bi-cyclic ring having from 0 to 3 heteroatoms selected from S, O and N, optionally substituted with one or more —(CH₂)_{q}—R₃ groups; or R₂ is a group of formula 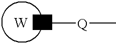
W is a 3 to 7 membered ring having one N heteroatom directly linked to Q and from 0 to 2 additional heteroatoms selected from the group consisting of S, SO, SO₂, O, N and NR′, wherein R′ is as above defined;
Q is a divalent group selected from CO, SO₂ and (CH₂)ₙ;
R₃ is selected, each independently, from the group consisting of alkyl, halogen, CF₃, OCF₃, NO₂, CN, C(═NR′)NR′R″, OR′, SR′, OCOR′, OCONR′R″, COCF₃, COR′, CO₂R′, CONR′R″, SO₂R′, SO₂NR′R″, NR′R″, NR′COR′, NR′COOR′, NR′CONR′R″, NR′SO₂R′, NR′SO₂NR′R″, wherein R′ and R″ are as above defined;
m is an integer from 1 to 4;
n is, each independently, 0, 1, or 2;
p is 1 or 2;
q is, each independently, 0 or an integer from 1 to 3;
r is an integer from 1 to 3;
   or isomers, tautomers, carriers, prodrugs, and pharmaceutically acceptable salts thereof.

In a preferred embodiment of the method described above, the disease caused by and/or associated with an altered protein kinase activity is selected from the group consisting of cancer, cell proliferative disorders, Alzheimer's disease, viral infections, auto-immune diseases and neurodegenerative disorders.

Specific types of cancer that may be treated include carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer and Kaposi's sarcoma.

In another preferred embodiment of the method described above, the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis glomerulonephritis and post-surgical stenosis and restenosis.

In addition, the method object of the present invention, also provides tumor angiogenesis and metastasis inhibition.

The present invention farther provides a compound represented by formula (I) 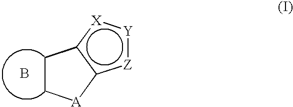
wherein
X, Y and Z, being part of an aromatic ring are selected, each independently, from the group consisting of N, NR₁, S, O and CR₁;
R₁ is selected from the group consisting of hydrido, lower alkyl, perfluorinated lower alkyl, heterocyclyl, CN, CO₂R′, COCF₃, COR′, CONR′R″, NR′R″, C(═NR′)NR′R″, CONHNH₂, CONHOR′, NHCOR′, CH₂NH₂, and CH₂NHCOR′; or R₁ may form, when part of Z or Y, a 5 to 7 membered ring together with the remaining of Y or Z, as per the formulae below 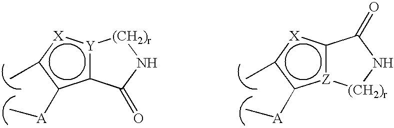
R′ and R″ are selected, each independently, from the group consisting of hydrido, hydroxy, alkyl, hydroxyalkyl, alkenyl, alkynyl, aryl, arylalkyl, heterocyclyl or heterocyclyl-alkyl;
B is an aromatic 5 or 6 membered ring having from 0 to 3 heteroatoms selected from, S, O and N;
A is selected from the group consisting of (CH₂)ₘ—, —(CH₂), —CH═CH—(CH₂)ₙ— and —(CR_{z}R_{y})ₚ—;
R_{z} and R_{y} are selected, each independently, from hydrido or lower alkyl; each of the X,Y,Z and B rings being optionally further substituted by one or more -L-R₂ groups, wherein L represents, each independently, a single bond, an alkylidene group or a divalent group selected from NH, NHCO, CONH, NHCONH, SO₂NH and NHSO₂;
R₂ is, each independently, hydrido, alkyl, 5 to 12 membered mono- or bi-cyclic ring having from 0 to 3 heteroatoms selected from S, O and N, optionally substituted with one or more —(CH₂)_{q}—R₃ groups; or R₂ is a group of formula 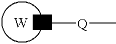
W is a 3 to 7 membered ring having one N heteroatom directly linked to Q and from 0 to 2 additional heteroatoms selected from the group consisting of S, SO, SO₂, O, N and NR′, wherein R′ is as above defined;
Q is a divalent group selected from CO, SO₂ and (CH₂)ₙ;
R₃ is selected, each independently, from the group consisting of alkyl, halogen, CF₃, OCF₃, NO₂, CN, C(═NR′)NR′R″, OR′, SR′, OCOR′, OCONR′R″, COCF₃, COR′, CO₂R′, CONR′R″, SO₂R′, SO₂NR′R″, NR′R″, NR′COR′, NR′COOR′, NR′CONR′R″, NR′SO₂R′, NR′SO₂NR′R″, wherein R′ and R″ are as above defined;
m is an integer from 1 to 4;
n is, each independently, 0, 1, or 2;
p is 1 or 2;
q is, each independently, 0 or an integer from 1 to 3;
r is an integer from 1 to 3;
   or isomers, tautomers, carriers, prodrugs, and pharmaceutically acceptable salts thereof.

Unless otherwise specified, when referring to the compounds of formula (I) per se as well as to any pharmaceutical composition thereof or to any therapeutic treatment comprising them, the present invention includes all of the hydrates, solvates, complexes and prodrugs of the compounds of this invention. Prodrugs are any covalently bonded compounds, which release the active parent drug according to formula (I) in vivo.

If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in formula (I) or any sub-formula thereof is independent of its meaning, or any other substituents meaning, at any other occurrence, unless specified otherwise.

In the present description, unless otherwise specified, within the X, Y, Z ring, each of X, Y and Z can be independently selected, as formerly indicated, among N, NR₁, S, O and CR₁, the penta-atomic ring so defined being an aromatic ring.

The term aromatic ring does not need any further clarification as it refers to any ring which can be conventionally defined as aromatic, such a term being widely used in organic chemistry.

Non limiting examples of X, Y, Z aromatic rings according to the invention are, for instance, thiophene, furan, furazan, pyrrole, pyrazole, imidazole, thiazole, isothiazole, oxazole or isoxazole.

When one or more of X, Y and Z are represented by NR₁ and/or CR₁ groups, the said ring is specifically substituted by R₁ groups, as above indicated.

With the term hydrido it is intended a single hydrogen atom (H); this hydrido radical may be attached, for example, to an oxygen atom to form a hydroxyl radical or two hydrido radicals may be attached to a carbon atom to form a methylene (—CH₂—) radical.

With the term lower alkyl group we intend any straight or branched alkyl group with from 1 to 6 carbon atoms such as, for instance, methyl, ethyl, n-propyl, isopropyl, n-butyl, isobutyl, sec-butyl, tert-butyl, n-pentyl, n-hexyl, and the like.

Pefluorinated lower alkyl groups stand for the above lower alkyl groups being further substituted in any of the free positions, at the same or different carbon atom, by more than one fluorine atoms. Non limiting examples of perfluorinated alkyl groups are, for instance, trifluoromethyl, 2,2,2-trifluoroethyl, 1,2-difluoroethyl, 1,1,1,3,3,3-hexafluoropropyl-2-yl, and the like.

Unless otherwise specified in the present description, with the term heterocyclyl we intend any 5 or 6 membered heterocyclic radical with from 1 to 3 heteroatoms selected among N, O and S. If not specifically noted otherwise, the said heterocyclic moieties may comprise saturated, partly unsaturated and fully unsaturated heterocycles; these latter, clearly referable to as aromatic heterocycles, are also conventionally known as heteroaromatic or heteroaryl rings. Non limiting examples of the said heterocycles of the invention are, for instance, thiophene, furan, furazan, pyran, pyrrole, imidazole, pyrazole, thiazole, isothiazole, oxazole, isoxazole, pyridine, pyrazine, pyrimidine, pyridazine, pyrrolidine, pyrroline, imidazolidine, imidazoline, pyrazolidine, pyrazoline, piperidine, piperazine, morpholine, and the like.

With the term hydroxyalkyl we intend any of the above straight or branched lower alkyl radicals having from one to six carbon atoms, any one of which may be substituted with one or more hydroxyl radicals.

With the term halogen atom, optionally referable to as “halo” group, herewith intended are fluorine, chlorine, bromine and iodine atoms.

With the term alkenyl or alkynyl we intend any of the aforementioned lower alkyl groups with from 2 to 6 carbon atoms, bearing a double or triple bond. Non limiting examples of alkenyl or alkynyl groups are thus, for instance, vinyl, allyl, 1-propenyl, isopropenyl, 1-butenyl, 2-butenyl, 3-butenyl, 2-pentenyl, 1-hexenyl, ethynyl, 2-propynyl, 4-pentynyl, and the like.

With the term aryl we intend, unless otherwise specified, any aromatic ring hence including carbocyclic or 5 or 6 membered heterocyclic rings with from 1 to 3 heteroatoms selected among N, O and S. Non limiting examples of aryl groups are thus phenyl, furyl, thienyl, pyrrolyl, imidazolyl, pyrazolyl, thiazolyl, isothiazolyl, oxazolyl, isoxazolyl, pyridyl, pyrazinyl, pyrimidinyl, pyridazinyl, and the like.

With the terms arylalkyl or heterocyclyl-alkyl groups we intend any of the above groups being defined according to the single moieties from which they derive. More particularly, arylalkyl and heterocyclyl-alkyl groups stand for the above alkyl groups further substituted by aryl or heterocyclyl groups, respectively, these latter being as above defined.

In the present description, unless otherwise specified, B represents a 5 to 6 membered aromatic ring, as formerly indicated, having from 0 to 3 heteroatoms selected from N, O and S. From the above it is clear to the skilled man that B may comprise phenyl, as a 6 membered aromatic ring with 0 heteroatoms, as well as any other 5 or 6 membered aromatic heterocycle with from 1 to 3 heteroatoms, as above defined.

In formula (I), A represents a divalent linker joining X, Y, Z ring with B ring. According to the meanings provided to A, therefore, it may represent a straight or branched alkylidene group being optionally unsaturated [e.g. —(CR_{z}R_{y})ₚ— such as, for instance, —(CH₂)ₙ—CH═CH—(CH₂)ₙ—].

Apart from what above reported, both B and X, Y, Z rings may be optionally further substituted, each independently, by one or more L-R₂ groups, being the same or different. Substitutions may obviously occur in any of the free positions of both rings, by replacement of one or more hydrogen atoms, otherwise referred to as hydrido.

When referring to alkylidene, L may represent a saturated divalent hydrocarbon group, with from 1 to 6 carbon atoms such as, for instance, a —(CH₂)₁₋₆— group.

Unless otherwise specified, with the term 5 to 12-membered, either mono- or bi-cyclic ring system, with 0 to 3 heteroatoms among N, O and S, we intend any carbocyclic (e.g. 0 heteroatoms) or heterocyclic (e.g. 1 to 3 heteroatoms) ring, either saturated, partly unsaturated or fully unsaturated (e.g. aromatic) ring system. Unless otherwise defined, within the above bi-cyclic ring systems, each of the two ring units may be fused to each other or otherwise linked through a single bond.

Non limiting examples of the above carbocyclic ring systems include, for instance, cyclopentane, cyclopentene, cyclohexane, cyclohexene, cyclohexadiene, benzene, naphthalene and biphenylene.

Examples of the above heterocylic ring systems may typically include any of the aforementioned 5 or 6 membered, either saturated, partly unsaturated or fully unsaturated heterocycles (see examples above) which may be further condensed to, or linked through a single bond with, any of the aforementioned mono-cyclic carbocyclic or heterocyclic rings themselves.

Finally, when referring to the W ring, it represents a 3 to 7 membered heterocyclic ring at least containing a N nitrogen atom directly linked to Q, as set forth above.

The term “pharmaceutically acceptable salts” embraces salts commonly used to form alkali metal salts and to form addition salts of free acids or free bases. The nature of the salt is not critical, provided that it is pharmaceutically acceptable. Suitable pharmaceutically acceptable acid addition salts of compounds of the present invention may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic and sulfonic classes of organic acids, examples of which are formic, acetic, trifluoroacetic propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, phydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, cyclohexylaminosulfonic, algenic, hydroxybutyric, salicyclic, galactaric and galacturonic acid. Suitable pharmaceutically acceptable base addition salts of compounds of the present invention include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methyl-glucamine) and procaine. All of these salts may be prepared by conventional means from the corresponding compound of the present invention by reacting, for example, the appropriate acid or base.

A class of preferred compounds of the invention is represented by the derivatives of formula (Ia) 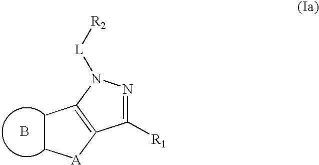
wherein B, R₁, L and R₂ are as above defined and A is selected from the group consisting of —CH₂—, —CH₂—CH₂—, —CH═CH— and —CH₂—C(CH₃)₂—, the B ring being optionally further substituted as above defined.

Another class of preferred compounds of the invention is represented by the derivatives of formula (Ib) 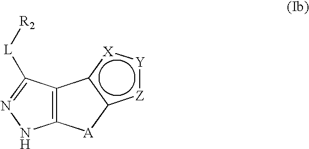
wherein X, Y, Z, L and R₂ are as above defined and A is selected from the group consisting of —CH₂—, —CH₂—CH₂—, —CH═CH— and —CH₂—C(CH₃)₂—, the X, Y, Z ring being optionally further substituted as above defined.

Another class of preferred compounds of the invention is represented by the derivatives of formula (Ic) 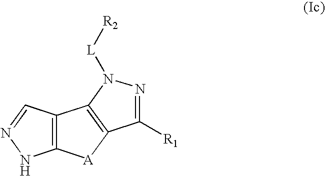
wherein R₁, L and R₂ are, each independently, as above defined, and A is selected from the group consisting of —CH₂—, —CH₂—CH₂—, —CH═CH— and —CH₂—C(CH₃)₂—.

Another class of preferred compounds of the invention is represented by the derivatives of formula (Id) 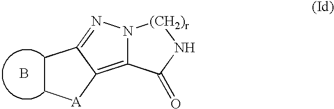
wherein r and B are as above defined, A is selected from the group consisting of —CH₂—, —CH₂—CH₂—, —CH═CH— and —CH₂—C(CH₃)₂—, and the B ring being optionally farther substituted as above defined.

Another class of preferred compounds of the invention is represented by the derivatives of formulae (Ie) or (If) 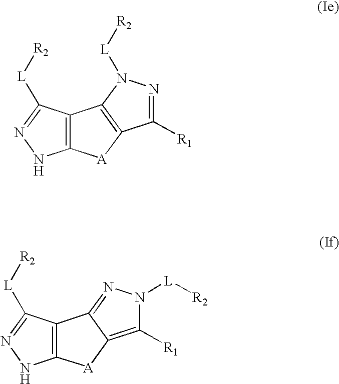
wherein L and R₂ are, each independently and the same or different in each occasion, as above defined; A is selected from the group consisting of —CH₂—CH₂—, —CH═CH— and —CH₂—C(CH₃)₂—; and R₁ is a group selected from NR′R″, CN, CO₂R′, COR′, CONR′R″, CONHOR′, CONHNH₂ and C(═NOH)NR′R″, wherein R′ and R″ are, the same or different, hydrido or alkyl.

Another class of preferred compounds of the invention is represented by the derivatives of formula (Ig) 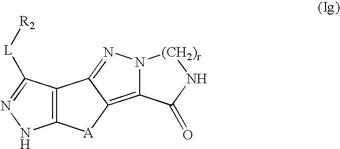
wherein L, R₂ and r are as above defined; and A is selected from the group consisting of —CH₂—CH₂—, —CH═CH— and —CH₂—C(CH₃)₂—.

Still more preferred, in any one of the above classes, are the derivatives of formula (I) wherein L is methylene or a single bond and R₂ is hydrido, phenyl or a 5 or 6 membered aromatic heterocycle having 1 or 2 heteroatoms selected among N, O and S, the said phenyl or heterocycle being optionally further substituted as above indicated.

Even more preferred are these latter derivatives of formula (I) wherein R₂, being optionally further substituted as above indicated, is selected from the group consisting of hydrido, phenyl, pyridyl, pyridazinyl or pyrimidinyl.

For a general reference to the specific compounds of formula (I) of the invention, and the pharmaceutically acceptable salts thereof, see the experimental section.

As set forth above, it is a further object of the present invention a process for preparing the compounds of formula (I) and the pharmaceutically acceptable salts thereof The said process can be conveniently described as set forth below according to Schemes I-VI.

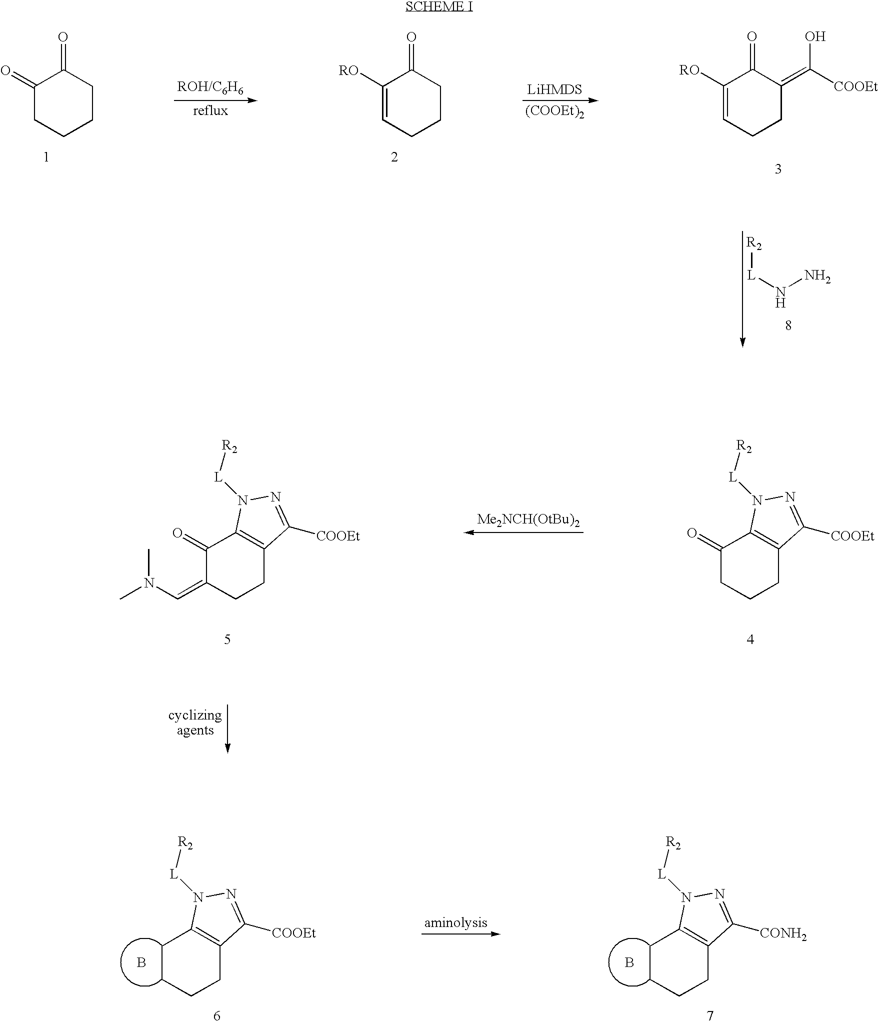

Scheme I describes the synthesis of the pyrazoles of formula (1) with fused heterocycles such as, for instance, substituted pyrimidine and pyrazole derivatives. In step one, 1,2-cyclohexanedione (1) was refluxed with alcohols such as methanol or ethanol in benzene to provide the desired enone (2). In step two, enone (2) was treated with a base such as lithium bistrimethylsilylamide, followed by condensation with diethyl oxalate to afford 1,3-diketone (3).

In step three, 1,3-diketone was allowed to react with a suitably substituted hydrazine of general formula (8) to form pyrazole (4).

In step four, pyrazole was treated with dimethylformamide di-tert-butyl acetal to give enaminone (5). In step five, enaminone was condensed with cyclizing agents such as hydrazine, guanidine, or thiourea derivatives to afford fused pyrazoles and pyrimidines (6).

In the final step, the ester was converted to amide (7) by treatment with ammonium hydroxide in methanol, at a temperature ranging from about 25° C. to about 70° C., in a sealed tube.

Hydrazines of general formula (8) are commercially available or can be obtained through synthetic procedures well described in the literature. For instance, aryl-hydrazines can be conveniently obtained from the corresponding anilines by diazotization, using sodium nitrite, or an alkyl nitrite, followed by catalytic or chemical reduction as described, for example, in J. Med. Chem., 36, 1529 (1993). In selected cases, aryl halides suitably activated with electron withdrawing groups can be converted to the corresponding arylhydrazines thorough displacement of the halogen atom with hydrazine or a carbazate, followed by hydrolysis of the protecting group, for instance as reported in J. Het. Chem., 25, 1543 (1988) or in Tetrah. Lett., 40 (18), 3543 (1999).

Alkyl-hydrazines can be obtained from alkyl-amines by treatment with hydroxylamine-O-sulfonic acid, for instance as described in JOC, 14, 813 (1949).

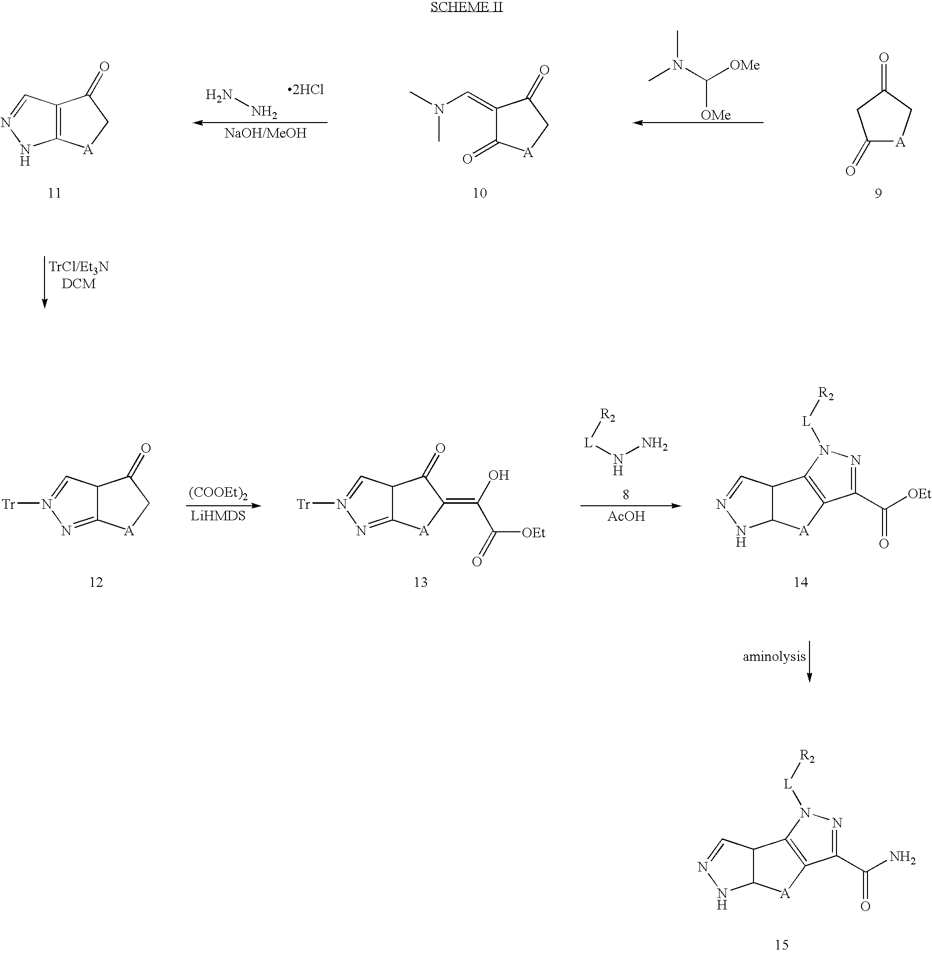

The synthetic pathway reported in Scheme II illustrates a procedure, alternative to Scheme I, for the preparation of derivatives of general formula (I) wherein A is preferably selected among —CH₂—, —CH₂—CH₂—, —CH₂—C(CH₃)₂—.

In step one, the cyclic diketone (9) was condensed with N,N-dimethylformamide dimethyl acetal to obtain the adduct (10), as described in Heterocycles, 32, 41 (1991). In step two, the adduct (10) was reacted with hydrazine dihydrochloride to obtain the intermediate (11), that was protected with trityl chloride (step three) to give the intermediate (12). After condensation with oxalyl chloride (step four), the diketoester (13) was allowed to react with a suitably substituted hydrazine (8) (step five) to form the dipyrazole (14). If a salified form of the hydrazine (8) is used (i.e. hydrochloride), the trityl protecting group is normally lost during the cyclization reaction. Optionally, diluted hydrochloric acid can be added to complete the deprotection, once the cyclization has occurred. In step six, the ester was then converted to the amide (15) by treatment with ammonium hydroxide in methanol, at a temperature ranging from about 25° C. to about 70° C., in a sealed tube.

The intermediate compound (11) wherein A is —CH₂— or —CH₂—CH₂—, as well as the intermediate compounds (12) and (13) wherein A is selected from —CH₂—, —CH₂—CH₂— and —CH₂—C(CH₃)₂— are novel and, hence, represent a further object of the present invention.

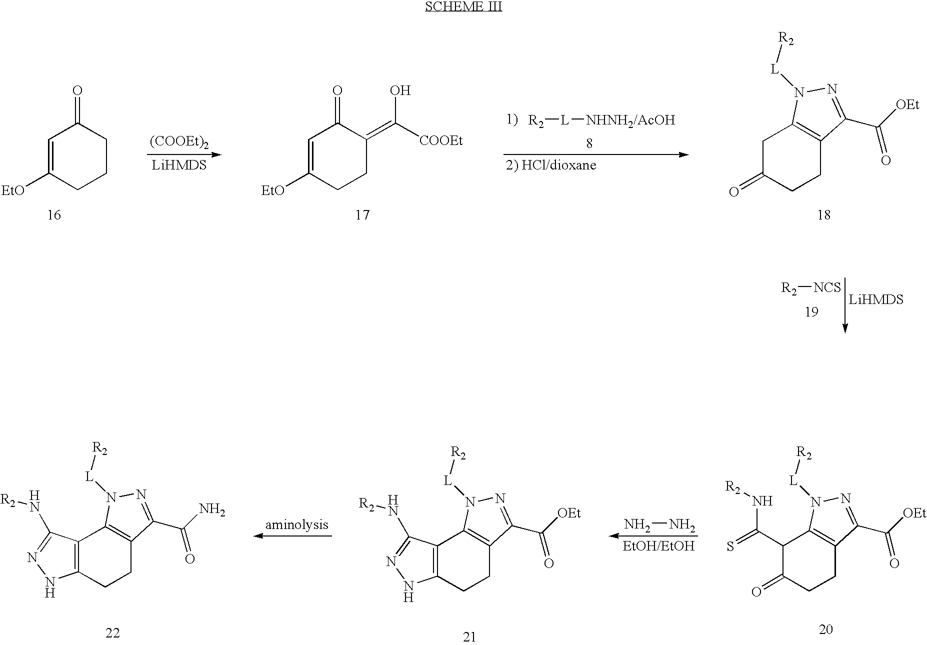

Scheme III illustrates the general synthetic procedure for the preparation of benzodipyrazole derivatives of general formula (I) wherein B is further substituted by a L-R₂ group wherein L is NH.

In step one the commercially available 3-ethoxy-cyclohex-2-enone (16) is condensed with diethyl oxalate to afford the diketoester (17), which is then reacted, in step two, with a suitably substituted hydrazine (8) to give the pyrazole derivative (18).

In step three the pyrazole (18) is treated in the presence of a base, such as lithium bistrimethylsilylamide, with a suitably substituted isothiocyanate (19) to afford the intermediate (20), which is then converted to the 3-aminobenzodipyrazole ester of formula (21). In the last step, the ester (21) is finally converted to the corresponding amide (22) under standard operative conditions. Isothiocyanates of general formula (19) are commercially available or can be obtained through synthetic procedures well described in the literature.

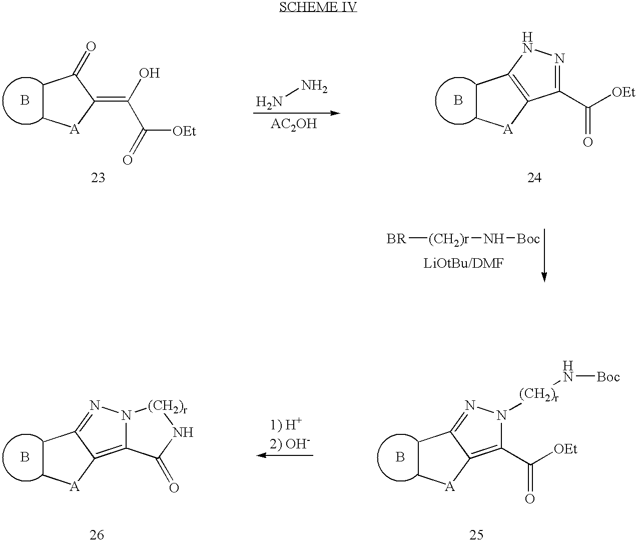

Scheme IV describes the general synthetic pathway to obtain compounds of general formula (I) wherein Y and Z are linked so as to form an additional lactamic ring and A is preferably selected from —CH₂—, —CH₂—CH₂— or —CH₂—C(CH₃)₂—. More generally, scheme IV can also be used to obtain compounds of general formula (I) wherein group L-R₂ is linked to Y. In the first step, the intermediate compound (23) is reacted with hydrazine to form the pyrazole derivative (24). This is then alkylated, in step two, using an alkyl halide bearing a protected amino group, for instance as tert-butoxy-carbonyl (BOC) amino group. In step three, after removal of the protecting group, the intermediate (25) is allowed to cyclize so to form the final compound (26) under standard operative conditions.

SCHEME V

| | |
|---|---|
| 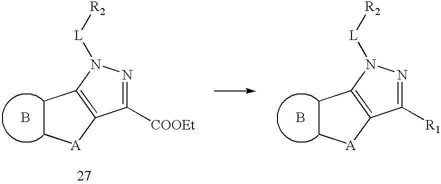 | 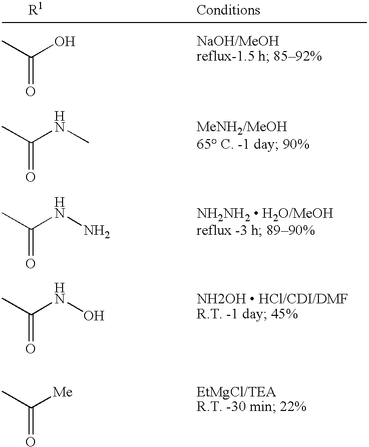 |
| 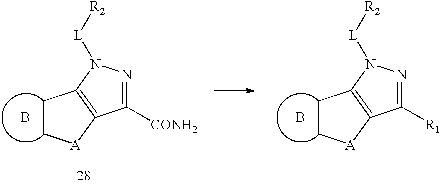 | 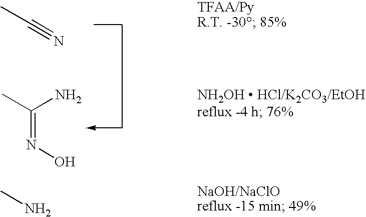 |

Scheme V refers to some examples describing the possibility of obtaining compounds of general formula (I), differently substituted in R₁. Preferably, A is selected from the group consisting —CH₂—, —CH₂—CH₂—, —CH₂—C(CH₃)₂— and —CH═CH—.

The above reactions of transformation are generally performed by properly reacting the alkoxycarbonyl group of the intermediate (27) or the aminocarbonyl group of the intermediate (28), each of which may be suitably protected. The transformations and related experimental conditions shown in scheme V, are readily apparent to one skilled in the art and are thus provided for exemplification purposes only, without limiting the scope of the invention.

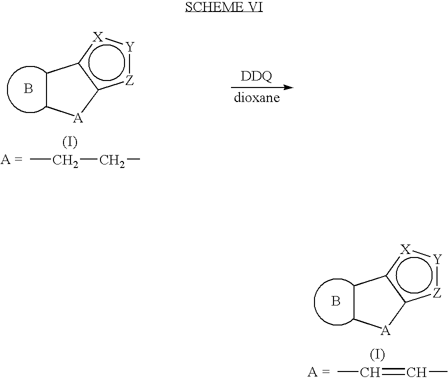

Synthetic scheme VI describes a general procedure for transforming the compounds of formula (I) wherein both B and X, A, Z rings are as defined in the above general formula and A is —CH₂—CH₂—, to the corresponding aromatic counterparts of general formula (I) wherein A is —CH═CH—. The oxidation of the central ring can be accomplished according to conventional techniques, for instance by using activated quinone derivatives, e.g. 2,3-dichloro-5,6-dicyano-1,4-benzoquinone or, alternatively, palladium on charcoal in a suitable solvent such as decalin, at high temperatures.

When preparing the compounds of formula (I) according to any variant of the process, which are all to be intended as within the scope of the present invention, optional functional groups within both the starting materials, the reagents or the intermediates thereof, and which could give rise to unwanted side reactions, need to be properly protected according to conventional techniques.

Likewise, the conversion of these latter into the free deprotected compounds may be carried out according to known procedures.

Pharmaceutically acceptable salts of the compounds of formula (I) or, alternatively, their free compounds from the salts thereof, my be all obtained according to conventional methods.

Any of the starting material within schemes I to VI and reactants thereof are known, or may be easily prepared according to known methods.

From all of the above, it is also clear to the skilled man that any compound of formula (I) of the invention may be prepared by working in analogy to what reported in anyone of schemes I to VI and, perhaps, by optionally providing any required modification to the above reactions, on a case by case. The said reactions are however known and conventionally adopted when preparing tricyclyc heterocyclic derivatives of formula (I) and substituted compounds thereof.

### PHARMACOLOGY

The compounds of formula (I) are active as protein kinase inhibitors and are therefore useful, for instance, to restrict the unregulated proliferation of tumor cells.

In therapy, they may be used in the treatment of various tumors, such as those formerly reported, as well as in the treatment of other cell proliferative disorders such as psoriasis, vascular smooth cell proliferation associated with atherosclerosis and post-surgical stenosis and restenosis and in the treatment of Alzheimer's disease.

The inhibiting activity of putative Cdk/Cyclin inhibitors and the potency of selected compounds was determined through a method of assay based on the use of the SPA technology (Amersham Pharmacia Biotech).

The assay consists of the transfer of radioactivity labelled phosphate moiety by the kinase to a biotinylated substrate. The resulting 33P-labelled biotinylated product is allowed to bind to streptavidin-coated SPA beads (biotin capacity 130 pmol/mg), and light emitted was measured in a scintillation counter.

Inhibition Assay of Cdk2/Cyclin A Activity

Kinase reaction: 4 μM in house biotinylated histone H1 (Sigma # H-5505) substrate, 10 μM ATP (0.1 microCi P³³γ-ATP), 4.21 ng Cdk2/Cyclin A complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 30 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate.

After 20 min. incubation for substrate capture, 100 μl 5M CsCl were added to allow statification of beads to the top of the plate and let stand 4 hours before radioactivity counting in the Top-Count instrument.

IC50 determination: inhibitors were tested at different concentrations ranging from 0.0015 to 10 μM. Experimental data were analyzed by the computer program GraphPad Prizm using the four parameter logistic equation:
y=bottom+(top−bottom)/(1+10\hat{ }((log IC50−x)*slope))
where x is the logarithm of the inhibitor concentration, y is the response; y starts at bottom and goes to top with a sigmoid shape.
Ki Calculation:

Experimental method: Reaction was carried out in buffer (10 mM Tris, pH 7.5, 10 mM MgCl₂, 0.2 mg/ml BSA, 7.5 mM DTT) containing 3.7 nM enzyme, histone and ATP (constant ratio of cold/labeled ATP 1/3000). Reaction was stopped with EDTA and the substrate captured on phosphomembrane (Multiscreen 96 well plates from Millipore). After extensive washing, the multiscreen plates are read on a top counter. Control (time zero) for each ATP and histone concentrations was measured.

Experimental design: Reaction velocities are measured at different four ATP, substrate (histone) and inhibitor concentrations. An 80-point concentration matrix was designed around the respective ATP and substrate Km values, and the inhibitor IC50 values (0.3, 1, 3, 9 fold the Km or IC50 values). A preliminary time course experiment in the absence of inhibitor and at the different ATP and substrate concentrations allow the selection of a single endpoint time (10 min) in the linear range of the reaction for the Ki determination experiment.

Kinetic parameter estimates: Kinetic parameters were estimated by simultaneous nonlinear least-square regression using [Eq.1] (competitive inhibitor respect to ATP, random mechanism) using the complete data set (80 points):

$$v = \frac{Vm \cdot A \cdot B}{α \cdot Ka \cdot Kb + α \cdot Ka \cdot B + a \cdot Kb \cdot A + A \cdot B + α \cdot \frac{Ka}{Ki} \cdot I \cdot ( Kb + \frac{B}{β} )} \qquad [ Eq . 1 ]$$

where A=[ATP], B=[Substrate], I=[inhibitor], Vm=maximum velocity, Ka, Kb, Ki the dissociation constants of ATP, substrate and inhibitor respectively. α and β the cooperativity factor between substrate and ATP binding and substrate and inhibitor binding respectively.

In addition the selected compounds have been characterized on a panel of ser/threo kinases strictly related to cell cycle (Cdk2/Cyclin E, Cdk1/cyclin B1, Cdk5/p25, Cdk4/Cyclin D1), and also for specificity on MAPK, PKA, EGFR, IGF1-R, Aurora-2 and Akt.

Inhibition Assay of Cdk2/Cyclin E Activity

Kinase reaction: 10 μMin house biotinylated histone H1 (Sigma # H-5505) substrate, 30 μMATP (0.3 microCi P³³γ-ATP), 4 ng GST-Cdk2/Cyclin E complex, inhibitor in a final volume of 30 μl buffer (TRIS HCl 10 mM pH 7.5, MgCl₂ 10 mM, DTT 75 mM+0.2 mg/ml BSA) were added to each well of a 96 U bottom. After 60 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate.

After 20 min. incubation for substrate capture, 100 μl 5M CsCl were added to allow statification of beads to the top of the plate and let stand 4 hours before radioactivity counting in the Top-Count instrument.

IC50 determination: see above

Inhibition Assay of Cdk1/Cyclin B1 Activity

Kinase reaction: 4 μM in house biotinylated histone H1 (Sigma # H-5505) substrate, 20 μM ATP (0.2 microCi P³³γ-ATP), 3 ng Cdk1/Cyclin B complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 20 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate.

After 20 min. incubation for substrate capture, 100 μl 5M CsCl were added to allow statification of beads to the top of the Optiplate and let stand 4 hours before radioactivity counting in the Top-Count instrument.

IC50 determination: see above

Inhibition Assay of Cdk5/p25 Activity

The inhibition assay of Cdk5/p25 activity was performed according to the following protocol.

Kinase reaction: 10 μM biotinylated histone H1 (Sigma # H-5505) substrate, 30 μM ATP (0.3 microCi P³³7-ATP), 15 ng CDK5/p25 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 30 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate.

After 20 min. incubation for substrate capture, 100 μl 5M CsCl were added to allow statification of beads to the top of the plate and let stand 4 hours before radioactivity counting in the Top-Count instrument.

IC50 determination: see above

Inhibition Assay of Cdk4/Cyclin D1 Activity

Kinase reaction: 0.4 uM μM mouse GST-Rb (769-921) (# sc-4112 from Santa Cruz) substrate, 10 μM ATP (0.5 μCi P³³γ-ATP), 100 ng of baculovirus expressed GST-Cdk4/Cyclin D1, suitable concentrations of inhibitor in a final volume of 50 μl buffer (TRIS HCl 10 mM pH 7.5, MgCl₂ 10 mM, 7.5 mM DTT+0.2 mg/ml BSA) were added to each well of a 96 U bottom well plate. After 40 min at 37° C. incubation, reaction was stopped by 20 μl EDTA 120 mM.

Capture: 60 μl were transferred from each well to MultiScreen plate, to allow substrate binding to phosphocellulose filter. Plates were then washed 3 times with 150 μl/well PBS Ca⁺⁺/Mg⁺⁺ free and filtered by MultiScreen filtration system.

Detection: filters were allowed to dry at 37° C., then 100 μl/well scintillant were added and ³³P labeled Rb fragment was detected by radioactivity counting in the Top-Count instrument.

IC50 determination: see above

Inhibition Assay of MAPK Activity

Kinase reaction: 10 μM in house biotinylated MBP (Sigma # M-1891) substrate, 15 μM ATP (0.15 microCi P³³γ-ATP), 30 ng GST-MAPK (Upstate Biothecnology # 14-173), inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 30 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate.

After 20 min. incubation for substrate capture, 10.0 μl 5M CsCl were added to allow statification of beads to the top of the Optiplate and let stand 4 hours before radioactivity counting in the Top-Count instrument.

IC50 determination: see above

Inhibition Assay of PKA Activity

Kinase reaction: 10 μM in house biotinylated histone H1 (Sigma # H-5505) substrate, 10 μM ATP (0.2 microM P³³γ-ATP), 0.45 U PKA (Sigma # 2645), inhibitor in a final volume of 30 μL buffer (TRIS HCl 10 mM pH 7.5, MgCl₂ 10 mM, DTT 7.5 mM+0.2 mg/ml BSA) were added to each well of a 96 U bottom. After 90 min at r.t. incubation, reaction was stopped by 100 μl PBS+32 nM EDTA+0.1% Triton X-100+500 μM ATP, containing 1 mg SPA beads. Then a volume of 110 μl is transferred to Optiplate.

After 20 min. incubation for substrate capture, 100 μl 5M CsCl were added to allow statification of beads to the top of the Optiplate and let stand 4 hours before radioactivity counting in the Top-Count instrument.

IC50 determination: see above

Inhibition Assay of EGFR Activity

Kinase reaction: 10 μM in house biotinylated MBP (Sigma # M-1891) substrate, 2 μM ATP (0.04 microCi P³³γ-ATP), 36 ng insect cell expressed GST-EGFR, inhibitor in a final volume of 30 μl buffer (Hepes 50 mM pH 7.5, MgCl₂ 3 mrM, MnCl₂ 3 mM, DTT 1 mM, NaVO₃ 3 μM+0.2 mg/ml BSA) were added to each well of a 96 U bottom. After 20 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate.

After 20 min. incubation for substrate capture, 100 μl 5M CsCl were added to allow statification of beads to the top of the Optiplate and let stand 4 hours before radioactivity counting in the Top-Count instrument.

IC50 determination: see above

Inhibition Assay of IGF1-R Activity

The inhibition assay of IGF1-R activity was performed according to the following protocol.

Kinase reaction: 10 μM biotinylated MBP (Sigma cat. # M-1891) substrate, 0-20 μM inhibitor, 6 μM ATP, 1 microCi ³³P-ATP, and 22.5 ng GST-IGF1-R (pre-incubated for 30 min at room temperature with cold 60 μM cold ATP) in a final volume of 30 μl buffer (50 mM HEPES pH 7.9, 3 mM MnCl₂, 1 mM DTT, 3 μM NaVO₃) were added to each well of a 96 U bottom well plate. After incubation for 35 min at room temperature, the reaction was stopped by addition of 100 oil PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

Inhibition Assay of Aurora-2 Activity

Kinase reaction: 8 μM biotinylated peptide (4 repeats of LRRWSLG), 10 μM ATP (0.5 uCi P³³γ-ATP), 15 ng Aurora2, inhibitor in a final volume of 30 μl buffer (HEPES 50 mM pH 7.0, MgCl₂ 10 nM, 1 mM DTT, 0.2 mg/ml BSA, 3 μM orthovanadate) were added to each well of a 96 U bottom well plate. After 30 minutes at room temperature incubation, reaction was stopped and biotinylated peptide captured by adding 100 μl of bead suspension.

Stratification: 100 μl of CsCl2 5 M were added to each well and let stand 4 hour before radioactivity was counted in the Top-Count instrument.

IC50 determination: see above

Inhibition Assay of Cdc7/dbf4 Activity

The inhibition assay of Cdc7/dbf4 activity was performed according to the following protocol.

The Biotin-MCM2 substrate is trans-phosphorylated by the Cdc7/Dbf4 complex in the presence of ATP traced with γ³³-ATP. The phosphorylated Biotin-MCM2 substrate is then captured by Streptavidin-coated SPA beads and the extent of phosphorylation evaluated by β counting.

The inhibition assay of Cdc7/dbf4 activity was performed in 96 wells plate according to the following protocol.

To each well of the plate were added:
10 μl substrate (biotinylated MCM2, 6 μM final concentration)
10 μl enzyme (Cdc7/Dbf4, 12.5 nM final concentration)
10 μl test compound (12 increasing concentrations in the nM to μM range to generate a dose-response curve)
10 μl of a mixture of cold ATP (10 μM final concentration) and radioactive ATP (1/2500 molar ratio with cold ATP) was then used to start the reaction which was allowed to take place at 37° C.

Substrate, enzyme and ATP were diluted in 50 mM HEPES pH 7.9 containing 15 mM MgCl₂, 2 mM DTT, 3 μM NaVO₃, 2 mM glycerophosphate and 0.2 mg/ml BSA. The solvent for test compounds also contained 10% DMSO.

After incubation for 20 minutes, the reaction was stopped by adding to each well 100 μl of PBS pH 7.4 containing 50 mM EDTA, 1 mM cold ATP, 0.1% Triton X100 and 10 mg/ml streptavidin coated SPA beads.

After 15 minutes of incubation at room temperature to allow the biotinylated MCM2-streptavidin SPA beads interaction to occur, beads were trapped in a 96 wells filter plate (Unifilter^{R} GF/B™) using a Packard Cell Harvester (Filtermate), washed with distilled water and then counted using a Top Count (Packard).

Counts were blank-subtracted and then the experimental data (each point in triplicate) were analyzed for IC50 determination using a non-linear regression analysis (Sigma Plot).

The compounds of formula (I) of the present invention, suitable for administration to a mammal, e.g. to humans, can be administered by the usual routes and the dosage level depends upon the age, weight, conditions of the patient and the administration route.

For example, a suitable dosage adopted for oral administration of a compound of formula (I) may range from about 10 to about 500 mg pro dose, from 1 to 5 times daily.

The compounds of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or by intravenous and/or intrathecal and/or intraspinal injection or infusion.

In addition, the compounds of the invention can be administered either as single agents or, alternatively, in combination with known anticancer treatments such as radiation therapy or chemotherapy regimen in combination with cytostatic or cytotoxic agents, antibiotic-type agents, alkylating agents, antimetabolite agents, hormonal agents, immunological agents, interferon-type agents, cyclooxygenase inhibitors (e.g. COX-2 is inhibitors), metallomatrixprotease inhibitors, telomerase inhibitors, tyro sine kinase inhibitors, anti-growth factor receptor agents, anti-HER agents, anti-EGFR agents, anti-angiogenesis agents, farnesyl transferase inhibitors, ras-raf signal transduction pathway inhibitors, cell cycle inhibitors, other cdks inhibitors, tubulin binding agents, topoisomerase I inhibitors, topoisomerase II inhibitors, and the like.

As an example, the compounds of the invention can be administered in combination with one or more chemotherapeutic agents such as, for instance, exemestane, formestane, anastrozole, letrozole, fadrozole, taxane, taxane derivatives, encapsulated taxanes, CPT-11, camptothecin derivatives, anthracycline glycosides, e.g., doxorubicin, idarubicin, epinibicin, etoposide, navelbine, vinblastine, carboplatin, cisplatin, estramustine phosphate, celecoxib, tamoxifeln, raloxifen, Sugen SU-5416, Sugen SU-6668, Herceptin, and the like, optionally within liposomal formulations thereof.

If formulated as a fixed dose, such combination products employ the compounds of this invention within the dosage range described above and the other pharmaceutically active agent within the approved dosage range.

Compounds of formula (I) may be used sequentially with known anticancer agents when a combination formulation is inappropriate.

The present invention also includes pharmaceutical compositions comprising a compound of formula (I) or a pharmaceutically acceptable salt thereof in association with a pharmaceutically acceptable excipient (which can be a carrier or a diluent).

The pharmaceutical compositions containing the compounds of the invention are usually prepared following conventional methods and are administered in a pharmaceutically suitable form.

For example, the solid oral forms may contain, together with the active compound, diluents, e.g. lactose, dextrose, saccharose, sucrose, cellulose, corn starch or potato starch; lubricants, e.g. silica, talc, stearic, magnesium or calcium stearate, and/or polyethylene glycols; binding agents, e.g. starches, arabic gum, gelatin, methylcellulose, carboxymethylcellulose or polyvinyl pyrrolidone; disaggregating agents, e.g. a starch, alginic, alginates or sodium starch glycolate; effervescing mixtures; dyestuffs; sweeteners; wetting agents such as lecithin, polysorbates, laurylsulfates; and, in general, non-toxic and pharmacologically inactive substances used in pharmaceutical formulations. Said pharmaceutical preparations may be manufactured in known manner, for example, by means of mixing, granulating, tabletting, sugar-coating, or film-coating processes.

The liquid dispersions for oral administration may be e.g. syrups, emulsions and suspensions.

The syrups may contain as carrier, for example, saccharose or saccharose with glycerin and/or mannitol and/or sorbitol.

The suspensions and the emulsions may contain as carrier, for example, a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions may contain as carrier, for example, sterile water or preferably they may be in the form of sterile, aqueous, isotonic saline solutions or they may contain as a carrier propylene glycol.

The suppositories may contain together with the active compound a pharmaceutically acceptable carrier, e.g. cocoa butter, polyethylene glycol, a polyoxyethylene sorbitan fatty ester surfactant or lecithin.

The following examples are herewith intended to better illustrate the present invention without posing any limitation to it.

### EXAMPLE 1

### Ethyl 1-(4-methoxyphenyl)-1,4,5,6-tetrahydropyrazolo[3,4-e]indazole-3-carboxylate

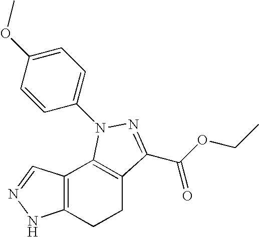

Step 1: Hydrazine hydrochloride (6.28 g, 59.8 mmols) was suspended in methanol (100 ml) and treated with 2N NaOH solution (90 ml, 3 eq). 2-[(dimethylamino)methylene]-cyclohexane-1,3-dione (10 g, 59.8 mmols) was then added and the mixture was kept at 80° C. for 3 hours. After cooling, the mixture was neutralised with N HCl and evaporated to dryness. The solid was extracted with ethyl acetate (100 ml×3) at 50° C. The extracts were collected and evaporated to give pure 1,5,6,7-tetrahydro-4H-indazol-4-one (8.03 g, Y=98%) as a yellow crystalline solid.

¹H NMR (CDCl₃/300 MHz) 2.16 (m, 2H); 2.52 (m, 2H); 2.90 (t, 2H); 8.00 (s, 1H).

Step 2: To a suspension of 1,5,6,7-tetrahydro-4H-indazol-4-one (8 g, 58.75 mmols) and trityl chloride (18.02 g, 64.64 mmols) in dichloromethane (160 ml), triethylamine (9.8 ml, 70.50 mmols) was added dropwise. The reaction was slightly exothermic.

After stirring overnight, the organic layer was washed with water, dried over MgSO4 and evaporated to dryness. The crude material was taken up with hexane, kept under vigorous stirring for 15 minutes and filtered on buchner to give 2-trityl-2,5,6,7-tetrahydro-4H-indazol-4-one (21 g, Y=94%).

¹H NMR (CDCl₃/300 MHz) 2.14 (m, 2H); 2.48 (m, 2H); 2.89 (m, 2H); 7.13 (m, 6H); 7.32 (m, 9H); 7.87 (s, 1H).

Step 3: To a suspension of 2-trityl-2,5,6,7-tetrahydro-4H-indazol-4-one (20 g, 52.84 mmols) and ethyl oxalate (7.88 ml, 58.13 mmols) in ethyl ether (150 ml), lithium bis(trimethylsilyl)amide 1M in THF (56.54 ml) was added dropwise. The slurry was stirred overnight, poured into a 20% NaH₂PO₄ solution (200 ml) and extracted with ethyl acetate. The extracts were washed with brine, dried over MgSO₄ and evaporated to dryness. The residue was taken up with ethanol and filtered to give ethyl oxo(4-oxo-2-trityl-4,5,6,7-tetrahydro-2H-indazol-5-yl)acetate as a pink solid (23.5 g, Y=93%).

¹H NMR (CDCl₃/300 MHz) 1.40 (t, 3H); 2.86 (m, 2H); 3.07 (m, 2H); 4.35 (q, 2H); 7.14 (m, 6H), 7.33 (m, 9H); 7.91 (s, 1H).

Step 4: A suspension of ethyl oxo(4-oxo-2-trityl-4,5,6,7-tetrahydro-2H-indazol-5-yl)acetate (400 mg, 0.84 mmols) and (4-methoxyphenyl)-hydrazine hydrochloride (164 mg, 0.94 mmols) in acetic acid (4 ml) was stirred at 65° C. for 3 hours. After cooling, the resulting suspension was filtered on buchner and washed, in sequence, with acetic acid, ethyl ether and water to obtain ethyl 1-(4-methoxyphenyl)-1,4,5,6-tetrahydropyrazolo[3,4-e]indazole-3-carboxylate (230 mg, Y=81%) as white solid.

¹H NMR (DMSO-d₆/400 MHz) 1.25 (t, 3H, 2.83-3.15 (m, H); 3.82 (s, 3H); 4.23 (q, 2H); 7.16 (d, 2H); 7.46 (d, 2H).

By working according to an analogous procedure, the following compounds were prepared:
Ethyl 1-[4-(aminosulfonyl)phenyl]-1,4,5,6-tetrahydropyrazolo[3,4-e]indazole-3-carboxylate

¹H NMR (DMSO-d₆/400 MHz) 1.3 (t, 3H); 2.8-3.1 (2d, 4H); 4.3 (q, 2H); 7.35 (s, 1H); 7.5 (d, 2H); 7.8-8.1 (2d, 4H);
Ethyl 1-{4-[(methylamino)sulfonyl]phenyl}1-1,4,5,6-tetrahydropyrazolo[3,4-e]indazole-3-carboxylate

¹H NMR (DMSO-d₆/400 MHz) 1.26 (t, 3H); 2.43 (ni, 3H); 2.85-3.09 (m, 4H); 4.31 (q, 2H); 7.39 (bs, 1H); 7.58 (q, 1H); 7.87 (d, 2H); 7.97 (d, 2H);
Ethyl 1-{4-[(butylamino)sulfonyl]phenyl}-1,4,5,6-tetrahydropyrazolo[3,4-e]indazole-3-carboxylate

¹H NMR (DMSO-d₆/400 MHz) 0.79 (t, 3H); 1.13-1.39 (m, 7H); 2.79 (q, 2H); 2.83 (t, 2H); 3.07 (t, 2H); 4.31 (q, 2H); 7.32 (s, 1H); 7.71 (t, 1H); 7.85 (d, 2H); 7.88 (d, 2H);
Ethyl 1-{4-[(dimethylamino)sulfonyl]phenyl}-1,4,5,6-tetrahydropyrazolo[3,4-e]indazole-3-carboxylate

¹H NMR (DMSO-d₆/400 MHz) 1.25 (t, 3H); 2.62 (s, 6H); 2.89 (t, 2H); 3.07 (t, 2H); 4.26 (q, 2H); 7.41 (s, 1H); 7.89-7.96 (m, 4H);
Ethyl 1-{4-[(diprop-2-ynylamino)sulfonyl]phenyl}-1,4,5,6-tetrahydropyrazolo[3,4-e]indazole-3-carboxylate

¹H NMR (DMSO-d₆/400 MHz) 1.25 (t, 3H); 2.85-3.1 (m, 4H); 3.2 (s, 2H, 4.18 (d, 4H); 4.35 (q, 2H); 7.35 (s, 1H); 7.89-8.1 (2d, 4H);
Ethyl 1-[4-(anilinosulfonyl)phenyl]-1,4,5,6-tetrahydropyrazolo[3,4-e]indazole-3-carboxylate

¹H NMR (DMSO-d₆/400 MHz) 1.24 (t, 3H); 2.86 (t, 2H); 3.04 (t, 2H); 4.29 (q, 2H); 7.01-7.25 (m, 6H); 7.79 (d, 2H); 7.85 (d, 2H);
Ethyl 1-[4-(methylsulfonyl)phenyl]-1,4,5,6-tetrahydropyrazolo[3,4-e]indazole-3-carboxylate;

¹H NMR (DMSO-d₆/400 MHz) 1.31 (t, 3H); 2.89 (t, 2H); 3.07 (t, 2H); 4.31 (q, 2H); 7.40 (s, 1H); 7.91 (d, 2H); 8.12 (d, 2H);
Ethyl 1-(4-{[(2-hydroxypropyl)amino]sulfonyl}phenyl)-1,4,5,6-tetrahydropyrazolo-[3,4-e]indazole-3-carboxylate

¹H NMR (DMSO-d₆/400 MHz) 1.01 (d, 3H); 1.25 (t, 3H); 2.75 (m, 2H); 2.85-3.1 (2 t, 4H); 3.6 (dd, 1H); 4.35 (dd, 1H); 7.35 (s, 1H); 7.7 (t, 1H); 7.85-8.05 (2d, 4H);
Ethyl 1-[4-(aminocarbonyl)phenyl]-1,4,5,6-tetrahydropyrazolo[3,4-e]indazole-3-carboxylate

¹H NMR (DMSO-d₆/400 MHz) 1.31 (t, 3H); 2.88 (t, 2H); 3.07 (t, 2H); 4.31 (q, 2H); 7.21 (bs, 1H); 7.43 (s, 1H); 7.69 (d, 2H); 8.07 (d, 2H); 8.19 (s, 1H);
Ethyl 1,4,5,6-tetrahydropyrazolo[3,4-e]indazole-3-carboxylate

¹H NMR (DMSO-d₆/400 MHz) 1.3 (t, 3H); 2.8-3.05 (2t, 4 H); 4.3 (m, 2H); 7.7 (bs, 1H);
Ethyl 1-phenyl-1,4,5,6-tetrahydropyrazolo[3,4-e]indazole-3-carboxylate

¹H NMR (DMSO-d₆/400 MHz) 1.3 (t, 3H); 2.86 (m, 2H); 3.07 (m, 2H); 4.29 (q, 2H); 7.47 (s, 1H); 7.47 (s, 1H); 7.54-7.60 (m, 5H);
Ethyl 1-(4-fluorophenyl)-1,4,5,6-tetrahydropyrazolo[3,4-e]indazole-3-carboxylate

¹H NMR (DMSO-d₆/400 MHz) 1.3 (t, 3H); 2.85-3.1 (m, 4H); 4.3 (q, 2H); 7.2 (s, 1H); 7.4-7.7 (m, 4H);
Ethyl 1-(4-bromophenyl)-1,4,5,6-tetrahydropyrazolo[3,4-e]indazole-3-carboxylate

¹H NMR (DMSO-d₆/400 MHz) 1.30 (t, 3H), 2.88 (t, 2H), 3.04 (t, 2H), 4.29 (q, 2H); 7.27 (s, 1H); 7.57 (d, 2H); 7.78 (d, 2H);
Ethyl 1-(4-methylphenyl)-1,4,5,6-tetrahydropyrazolo[3,4-e]indazole-3-carboxylate

¹H NMR (DMSO-d₆/400 MHz) 1.3 (t, 3H); 2.4 (s, 3H); 2.8-3.1 (2t, 4H); 4.3 (q, 2H); 7.1 (bs, 1H); 7.4-7.5 (2d, 4H);
Ethyl 1-(4-chlorophenyl)-1,4,5,6-tetrahydropyrazolo[3,4-e]indazole-3-carboxylate

¹H NMR (DMSO-d₆/400 MHz) 1.3 (t, 3H); 2.8-3.1 (m, 4H); 4.3 (q, 2H); 7.35 (bs, 1H); 7.65 (s, 4H);
Ethyl 1-(4-cyanophenyl)-1,4,5,6-tetrahydropyrazolo[3,4-e]indazole-3-carboxylate

¹H NMR (DMSO-d₆/400 MHz) 1.31 (t, 3H); 2.88 (m, 2H); 3.05 (m, 2H); 4.31 (q, 2H); 7.25 (s, 1H); 7.85 (d, 2H); 8.06 (d, 2H);
Ethyl 1-(4-nitrophenyl)-1,4,5,6-tetrahydropyrazolo[3,4-e]indazole-3-carboxylate

¹H NMR (DMSO-d₆/400 MHz) 1.31 (t, 3H); 2.89 (m, 2H), 3.05 (t, 2H); 4.32 (q, 2H); 7.47 (s, 1H); 7.93 (d, 2H); 8.43 (d, 2H);
Ethyl 1-[4-(trifluoromethyl)phenyl]-1,4,5,6-tetrahydropyrazolo[3,4-e]indazole-3-carboxylate

¹H NMR (DMSO-d₆/400 MHz) 1.31 (t, 3H); 2.87 (m, 2H); 3.09 (M, 2H); 4.32 (q, 2H); 7.42 (bs, 1H); 7.47 (s, 1H); 7.87 (d, 2H); 7.96 (d, 2H);
Ethyl 1-benzyl-1,4,5,6-tetrahydropyrazolo[3,4-e]indazole-3-carboxylate

¹H NMR (DMSO-d₆/400 MHz) 1.3 (t, 3H); 2.8-3.05 (m, 4H); 4.2 (q, 2H); 5.5 (s, 1H); 7.1-7.25 (m, 5H);
Ethyl 1-(3-hydroxybenzyl)-1,4,5,6-tetrahydropyrazolo[3,4-e]indazole-3-carboxylate

¹H NMR (DMSO-d₆/400 MHz) 1.3 (t, 3H); 2.8-3.05 (2t, 4H); 4.25 (q, 2H); 5.4 (s, 2H); 6.5 (s, 1H); 6.7 (m, 2H); 7.1 (t, 1H); 7.8 (bs, 1H); 9.4 (s, 1H);
Ethyl 1-pyridin-2-yl-1,4,5,6-tetrahydropyrazolo[3,4-e]indazole-3-carboxylate

¹H NMR (DMSO-d₆/400 MHz) 1.3 (t, 3H); 2.8-3.1 (2t, 4H); 4.35 (q, 2H); 7.45 (t, 1H); 7.9-8.1 (d+t, 2H); 8.15 (bs, 1H); 8.6 (d, 1H);
Ethyl 1-(6-chloropyridazin-3-yl)-1,4,5,6-tetrahydropyrazolo[3,4-e]indazole-3-carboxylate

¹H NMR (DMSO-d₆/400 MHz) 1.35 (t, 3H); 2.9-3.1 (2t, 4H); 4.38 (dd, 2H); 8.1(s, 1H); 8.15-8.25 (2d, 2H);
Ethyl 1-[4-(trifluoromethyl)pyrimidin-2-yl]-1,4,5,6-tetrahydropyrazolo[3,4-e]indazole-3-carboxylate

¹H NMR (DMSO-d₆/400 MHz) 1.3 (t, 3H); 2.85-3.1 (2t, 4H); 4.35 (q, 2H); 8.15 (s, 1H); 8.2-9.4 (2d, 2H);
Ethyl 1-(3-methylphenyl)-1,4,5,6-tetrahydropyrazolo[3,4-e]indazole-3-carboxylate

¹H NM (DMSO-d₆/400 MHz) 1.3 (t, 33H); 2.4 (s, 3H); 2.8-3.1 (2t, 4H); 4.3 (q, 2H); 7.2 (bs, 1H); 7.35-7.5 (m, 4H);
Ethyl 1-(3-chlorophenyl)-1,4,5,6-tetrahydropyrazolo[3,4-e]indazole-3-carboxylate

¹H NMR (DMSO-d₆/400 MHz) 1.3 (t, 3H); 2.8-3.1 (2m, 4H); 4.3 (q, 2H); 7.3 (s, 1H); 7.6, 7.7 (2s, 4H);
Ethyl 1-(3-fluorophenyl)-1,4,5,6-tetrahydropyrazolo[3,4-e]indazole-3-carboxylate

¹H NMR (DMSO-d₆/400 MHz) 1.3 (t, 3H); 2.8-3.1 (2m, 4H); 4.3 (q, 2H); 7.3 (s, 1H); 7.5-7.7 (m, 4H);
Ethyl 4,4-dimethyl-1-(4-methylphenyl)-1,4,5,6-tetrahydropyrazolo[3,4-e]indazole-3-carboxylate

¹H NMR (DMSO-d₆/400 MHz) 1.29 (t, 3H); 1.33 (s, 6H); 2.40 (s, 3H); 2.73 (s, 2H); 4.28 (q, 2H); 7.02 (bs, 1H); 7.43 (dd, 4H); 12.05 (bs, 1H).
Ethyl 1-pyridin-3-yl-1,4,5,6-tetrahydropyrazolo[3,4-e]indazole-3-carboxylate

¹HNMR (DMSO-d₆/400 MHz); 1.3 (t, 3H); 2.85 (m, 2H); 3.15 (t, 2H); 4.31 (q, 2H); 7.21 (bs, 1H); 7.6 (m, 1H); 8.15 (d, 1H); 8.75 (d, 1H); 8.85 (s, 1H); 12.7 (bs, 1H)
Ethyl 1-[4-(acetylamino)phenyl]-1,4,5,6-tetrahydropyrazolo[3,4-e]indazole-3-carboxylate

¹HNMR (DMSO-d₆/400 MHz); 1.25 (t, 3H); 2.05 (s, 3H); 2.8-3.05 (2 t, 4H); 4.31 (q, 2H); 7.15 (s, 1H); 7.45 (d, 2H); 7.8 (d, 2H); 10.21 (s, 1H)
Ethyl 1-{4-[(4-methylpiperazin-1-yl)sulfonyl]phenyl)}-1,4,5,6-tetrahydropyrazolo[3,4-e]indazole-3-carboxylate

¹H NMR (DMSO-d₆/400 MHz); 1.30 (t, 3H); 2.40 (s, 3H); 2.65-3.30 (m, 12H); 4.31 (q, 2H); 7.33 (bs, 1H); 7.95 (m, 4H)
4-[3-(ethoxycarbonyl)-5,6-dihydropyrazolo[3,4-e]indazol-1 (4H)-yl]benzoic acid

¹H NMR (DMSO-d₆/400 MHz); 1.31 (t, 3); 2.89 (t, 2H); 3.07 (t, 3H); 4.31 (q, 2H); 7.34 (s, 1H); 7.76 (dd, 2H); 8.14 (dd, 2H)
Ethyl 1-[4-(trifluoromethoxy)phenyl]-1,4,5,6-tetrahydropyrazolo[3,4-e]indazole-3-carboxylate

¹H NMR (DMSO-d₆/400 MHz); 1.30 (t, 3H); 2.88 (bt, 2H); 3.06 (t, 2H); 4.29 (q, 2H); 7.26 (bs, 1H); 7.58 (bd, 2H); 7.75 (bd, 2H)
Ethyl 1-butyl-1,4,5,6-tetrahydropyrazolo[3,4-e]indazole-3-carboxylate

¹H NMR (DMSO-d₆/400 MHz); 0.85 (t, 3H); 1.28 (m, 5H); 1.71 (m, 2H); 2.80 (bt, 2H); 2.97 (bt, 2H); 4.25 (q, 2H); 8.04 (br, 1H)
Ethyl 1-(2,5-dimethylphenyl)-1,4,5,6-tetrahydropyrazolo[3,4-e]indazole-3-carboxylate

¹H NMR (DMSO-d₆/400 MHz); 1.29 (t, 3H); 1.91 (s, 3H); 2.33 (s, 3H); 2.85 (bt, 2H); 3.08 (bt, 2H); 4.27 (q, 2H); 6.64 (bs, 1H); 7.18 (bs, 1H); 7.34 (dd, 2H)
Ethyl 1-{4-[amino(imino)methyl]phenyl}-1,4,5,6-tetrahydropyrazolo[3,4-e]indazole-3-carboxylate hydrochloride

¹H NMR (DMSO-d₆/400 MHz); 1.31 (t, 3H); 2.90 (bt, 2H); 3.07 (b, 2H); 4.30 (q, 2H); 7.19 (s, 1H); 7.89 (d, 2H); 8.01 (d, 2H); 9.03 (bs, 2H); 9.44 (bs, 2H)
Ethyl 1-[4-(1H-imidazol-2-yl)phenyl]-1,4,5,6-tetrahydropyrazolo[3,4-e]indazole-3-carboxylate hydrochloride

¹H NMR (DMSO-d₆/400 MHz); 1.31 (t, 31H); 2.90 (t, 2H); 3.08 (t, 2H); 4.30 (q, 2H); 7.19 (s, 1H); 7.86 (s, 2H); 7.94 (d, 2H); 8.24 (d, 2H)
Ethyl 1-methyl-1,4,5,6-tetrahydropyrazolo[3,4-e]indazole-3-carboxylate

¹H NMR (DMSO-d₆/400 MHz); 1.31 (t, 3H); 2.82 (bt, 2H); 3.00 (bt, 2H); 3.98 (s, 3H); 4.27 (q, 2H); 8.13 (bs, 1H)

### EXAMPLE 2

### Ethyl 8-anilino-1-methyl-1,4,5,6-tetrahydropyrazolo[3,4-e]indazole-3-carboxylate

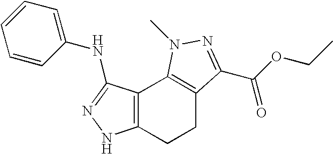

Step 1: A solution of 3-Ethoxy-cyclohex-2-enone (4.65 ml, 31.92 mmols) and diethyl oxalate (6.49 ml, 47.89 mmols) in anhydrous ethyl ether (50 ml) is treated dropwise with a 1M solution of lithium bis(trimethylsilyl)amide in tetrahydrofuran (47.9 ml, 47.9 mmols) under argon atmosphere. After standing at room temperature overnight, the mixture is poured into a 20% NaH2PO4 solution (150 ml) and extracted with ethyl acetate (100 ml×2). The organic exctracts are washed with brine, dried on Na2SO4 and evaporated to dryness to afford crude ethyl (4-ethoxy-2-oxocyclohex-3-en-1-yl)(oxo)acetate (8 g) as an orange oil which is used for the next step without further purification.

Step 2: (4-Ethoxy-2-oxo-cyclohex-3-enyl)-oxo-acetic acid ethyl ester (S g, 31.92 mmols theoretically) is treated with methylhydrazine (1.69 ml, 31.92 mmol) in EtOH (75 ml) and AcOH (5 ml) at room temperature. After 3 hours the solution was concentrated and the precipitate was collected to afford ethyl 6-ethoxy-1-methyl-4,5-dihydro-1H-indazole-3-carboxylate (7.59 g, Y=95%).

1H NMR (400 MHz, DMSO-D6) δ ppm 1.28 (t, J=7.07 Hz, 3H) 1.31 (t, J=7.01 Hz, 3H) 2.42 (t, J=8.60 Hz, 2H) 2.85 (t, J=8.66 Hz, 2H) 3.76 (s, 3H) 3.93 (td, J=7.07, 6.83 Hz, 2H) 4.23 (q, J=7.15 Hz, 2H) 5.74 (s, 1H)

Step 3: Ethyl 6-ethoxy-1-methyl-4,5-dihydro-1H-indazole-3-carboxylate (7.59 g, 30.36 mmols) was dissolved in dioxane (50 ml) and treated with HCl 2N (17 ml) overnight. The solution was concentrated, diluted with water and extracted with ethyl acetate. The organic phase was washed with brine, dried over sodium sulfate and evaporated to afford ethyl 1-methyl-6-oxo-4,5,6,7-tetrahydro-1H-indazole-3-carboxylate (5.6 g, Y=83%).

1H NMR (400 MHz, DMSO-D6) δ ppm 1.30 (t, J=7.07 Hz, 3H) 2.58 (t, J=6.89 Hz, 2H) 2.99 (t, J=6.89 Hz, 2H) 3.61 (s, 2H) 3.76 (s, 3H) 4.26 (q, J=7.07 Hz, 2H)

Step 4: A solution of ethyl 1-methyl-6-oxo-4,5,6,7-tetrahydro-1H-indazole-3-carboxylate (2.66 g, 12 mmol) in DMF (45 ml) was treated with lithium bis(trimethylsilyl)amide 1M in THF (13.2 ml, 13.2 mmol) at −40° C. After 15 minutes phenyl isothiocyanate (1.58 ml, 13.2 mmol) was added, dropwise.

After further 30 minutes the reaction mixture was treated with a 20% solution of sodium dihydrogen phosphate. The precipitate was filtered and washed with water to afford ethyl 7-(anilinocarbonothioyl)-1-methyl-6-oxo-4,5,6,7-tetrahydro-1H-indazole-3-carboxylate (3.11 g, Y=72%).

1H NMR (400 MHz, DMSO-D6) δ ppm 1.32 (t, J=7.07 Hz, 1H) 2.90 (m, 4H) 3.70 (s, 3H) 4.30 (q, J=7.07 Hz, 2H) 5.19 (s, 1H) 7.32 (t, J=7.44 Hz, 1H) 7.46 (t, J=7.93 Hz, 2H) 7.81 (d, J=7.56 Hz, 2H) 12.27 (s, 1H).

Step 5: A suspension of ethyl 7-(anilinocarbonothioyl)-1-methyl-6-oxo-4,5,6,7-tetrahydro-1H-indazole-3-carboxylate (3.10 g, 8.7 mmol) in EtOH (50 ml) and AcOH (0.5 ml) was treated with hydrazine hydrate (0.5 ml, 10.3 mmol) for 30 minutes under reflux. After cooling the white precipitated was collected by filtration to give ethyl 8-anilino-1-methyl-1,4,5,6-tetrahydropyrazolo[3,4-e]indazole-3-carboxylate (2.2 g, Y=75%).

1H NMR (400 MHz, DMSO-D6) δ ppm 1.30 (t, J=7.07 Hz, 1H) 2.90 (m, 4H) 3.83 (s, 3H) 4.25 (q, J=7.07 Hz, 2H) 7.32 (t, J=7.44 Hz, 1H) 7.46 (t, J=7.93 Hz, 2H) 7.81 (d, J=7.56 Hz, 2H) 8.07 (s, 1H) 12.79 (s, 1H)

By working analogously, the following compounds were prepared:
Ethyl 8-anilino-1-(2,2,2-trifluoroethyl)-1,4,5,6-tetrahydropyrazolo[3,4-e]indazole-3-carboxylate

¹H NMR (400 MHz, DMSO-D6) δ ppm 1.32 (t, J=7.07 Hz, 1H) 2.90 (m, 4H) 4.32 (q, J=7.07 Hz, 2H) 5.31 (m, 2H) 6.7 (m, 3H) 7.14 (m, 2H) 8.07 (s, 1H) 12.7 (s, 1H)
Ethyl 8-anilino-2-{2-[(tert-butoxy arbonyl)amino]ethyl}-2,4,5,6-tetrahydropyrazolo[3,4-e]indazole-3-carboxylate

1H NMR (400 MHz, DMSO-D6) δ ppm 1.36 (s, 9H), 1.36 (t, J=7.07 Hz, 1H) 2.82 (m, 2H) 3.05 (m, 2H) 3.33 (m, 1H), 4.33 (q, J=7.07 Hz, 2H) 4.50 (m, 2H), 6.77 (in, 2H) 7.21 (m, 3H) 7.45 (m, 2H) 12.11 (bs, 1H)
Ethyl 8-amino-1,4,5,6-tetrahydropyrazolo[3,4-e]indazole-3-carboxylate

1H NMR (400 MHz, DMSO-D6) δ ppm 1.34 (t, 3H), 2.87 (t, 2H), 3.05 (t, 2H), 4.32 (q, 2H)

### EXAMPLE 3

### 1-(4-methoxyphenyl)-1,4,5,6-tetrahydro-pyrazolo[3,4-e]indazole-3-carboxamide

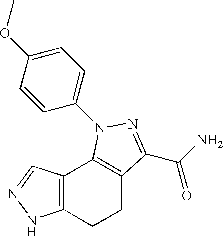

A suspension of ethyl 1-(4-methoxyphenyl)-1,4,5,6-tetrahydropyrazolo[3,4-e]indazole-3-carboxylate (200 mg; 0.59 mmols) in concentrated ammonium hydroxide (5 ml) and methanol (2.5 ml) was heated in a sealed tube at 65° C. for 8 hours. The mixture was then diluted with water and filtered to give 1-(4-methoxyphenyl)-1,4,5,6-tetrahydro-pyrazolo[3,4-e]indazole-3-carboxamide (137 mg; Y=75%) as a white solid.

¹H NMR (DMSO-d₆/400 MHz) 3.06 (t, 2H); 3.83 (s, 3H); 7.09-7.11 (m, 3H); 7.20 (s, 1H); 7.40 (s, 1H); 7.50 (m, 2H).

By working analogously, the following compounds were prepared:
1-[4-(aminosulfonyl)phenyl]-1,4,5,6-tetrahydropyrazolo[3,4-e]indazole-3-carboxamide

¹H NMR (DMSO-d₆/400 MHz) 2.8-3.2 (m, 4H); 7.3-7.5 (2s, 2H); 7.4 (s, 1H); 7.45 (d, 2H); 7.8-8.05 (2d, 4H);
1-{4-[(methylamino)sulfonyl]phenyl}-1,4,5,6-tetrahydropyrazolo[3,4-e]indazole-3-carboxamide

¹H NMR (DMSO-d₆/400 MHz) 2.47 (m, 3H, 2.81-3.07 (m, 4H); 7.25 (s, 2H); 7.41 (s, 1H); 7.45 (s, 2H); 7.54 (q, 1H); 7.88-7.97 (m, 4H);
1-{4-[(butylamino)sulfonyl]phenyl}-1,4,5,6-tetrahydropyrazolo[3,4-e]indazole-3-carboxamide

¹H NMR (DMSO-d₆/400 MHz) 0.79 (t, 3H); 1.20 (q, 2H); 1.40 (q, 2H); 2.79-2.90 (m, 4H); 3.06 (t, 2H); 7.23-7.55 (m, 3H); 7.65 (t, 1H); 7.85-7.98 (m, 4H);
1-{4-[(dimethylamino)sulfonyl]phenyl}-1,4,5,6-tetrahydropyrazolo[3,4-e]indazole-3-carboxamide

¹H NMR (DMSO-d₆/400 MHz) 2.66 (s, 6H); 3.15 (t, 2H); 7.20-7.50 (m, 3H); 7.95 (m, 4H);
1-{4-[(diprop-2-ynylamino)sulfonyl]phenyl}-1,4,5,6-tetrahydropyrazolo[3,4-e]indazole-3-carboxamide

¹H NMR (DMSO-d₆/400 MHz) 2.8-3.1 (2t, 4H); 3.25 (s, 2H); 4.20 (s, 4H); 7.25-7.45 (2s, 2H); 7.39 (s, 4H); 7.95-8.1 (2d, 4H);
1-[4-(anilinosulfonyl)phenyl]-1,4,5,6-tetrahydropyrazolo[3,4-e]indazole-3-carboxamide

¹H NMR (DMSO-d₆/400 MHz) 2.75-3.10 (m, 4H); 7.0-7.45 (m, 8H); 7.80 (d, 2H); 7.85 (d, 2H); 10.25 (s, 1H);
1-[4-(methylsulfonyl)phenyl]-1,4,5,6-tetrahydropyrazolo[3,4-e]indazole-3-carboxamide

¹H NMR (DMSO-d₆/400 MHz) 2.80 (m, 2H); 3.10 (m, 2H); 3.29 (s, 3H); 7.30 (s, 2H); 7.49 (s, 1H); 7.50 (s, 21H); 7.93 (d, 2H); 8.12 (d, 2H);
1-[4-(aminocarbonyl)phenyl]-1,4,5,6-tetrahydropyrazolo[3,4-e]indazole-3-carboxamide

¹H NMR (DMSO-d₆/400 MHz) 2.85 (m, 2H); 3.07 (t, 2H); 7.20 (s, 1H); 7.73 (d, 2H); 8.07 (d, 2H);
1-(4-{[(2-hydroxypropyl)amino]sulfonyl} phenyl)-1,4,5,6-tetrahydropyrazolo[3,4-e]indazole-3-carboxamide

¹H NMR (DMSO-d₆/400 MHz) 1.02 (d, 3H); 2.72 (m, 2H); 2.8-3.15 (m, 4H); 3.6 (m, 1H); 4.65 (d, 1H); 7.25-7.5 (2d, 2H); 7.4 (s, 1H); 7.65 (s, 1H); 7.85 (d, 2H); 8.01 (d, 2H);
1,4,5,6-tetrahydropyrazolo[3,4-e]indazole-3-carboxamide

¹H NMR (DMSO-d₆/400 MHz) 2.79 (bs, 2H); 2.98 (m, 2H); 7.28 (bs, 1H);
1-phenyl-1,4,5,6-tetrahydropyrazolo[3,4-e]indazole-3-carboxamide.

¹H NMR (DMSO-d₆/400 MHz) 2.82 (m, 2H); 3.09 (t, 2H); 7.20 (bs, 1H); 7.21 (bs, 1H); 7.43 bs, 1H); 7.45-7.63 (m, 5H);
1-(4-fluorophenyl)-1,4,5,6-tetrahydropyrazolo[3,4-e]indazole-3-carboxamide

¹H NMR (DMSO-d₆/400 MHz) 2.8-3.1 (m, 4H); 7.2 (m, 2H); 7.45 (m, 3H); 7.65 (m, 2H);
1-(4-bromophenyl)-1,4,5,6-tetrahydropyrazolo[3,4-e]indazole-3-carboxamide

¹H NMR (/400 MHz) 2.82 (bt, 2H); 3.28 (bt, 2H); 7.24 (bs, 1H); 7.36 (s, 1H); 7.46 (bs, 1H); 7.61 (d, 2H); 7.76 (d, 2H);
1-(4-nitrophenyl)-1,4,5,6-tetrahydropyrazolo[3,4-e]indazole-3-carboxamide

¹H NMR (/400 MHz) 2.85 (m, 2H); 3.06 (m, 2H); 7.32 (bs, 1H); 7.55 (bs, 1H); 7.57(s, 1H); 7.95 (d, 2H); 8.42 (d, 2H);
1-(4-methylphenyl)-1,4,5,6-tetrahydropyrazolo[3,4-e]indazole-3-carboxamide

¹H NMR (DMSO-d₆/400 MHz) 2.4 (s, 3H); 2.8-3.1 (m, 4H); 7.15 (s, 1H); 7.2-7.4 (2s, 2H); 7.35-7.45 (2d, 4H);
1-(4-chlorophenyl)-1,4,5,6-tetrahydropyrazolo[3,4-e]indazole-3-carboxamide

¹H NMR (DMSO-d₆/400 MHz) 2.8-3.1 (m, 4H); 7.2-7.45 (2s, 2H); 7.3 (s, 1H); 7.65 (m, 4H);
1-(4-cyanophenyl)-1,4,5,6-tetrahydropyrazolo[3,4-e]indazole-3-carboxamide

¹H NMR (DMSO-d₆/400 MHz) 2.85 (m, 2H); 3.06 (m, 2H); 7.30 (bs, 1H); 7.45 (bs, 1H); 7.55 (bs, 1H); 7.87 (d, 2H); 8.05 (d, 2H);
1-[4-(trifluoromethyl)phenyl]-1,4,5,6-tetrahydropyrazolo[3,4-e]indazole-3-carboxamide

¹H NMR (DMSO-d₆/400 MHz) 2.85 (bs, 2H); 3.09 (m, 2H); 7.25 (bs, 1H); 7.45 (bs, 1H); 7.51 (bs, 1H); 7.89 (d, 2H); 7.95 (d, 2H);
1-benzyl-1,4,5,6-tetrahydropyrazolo[3,4-e]indazole-3-carboxamide

¹H NMR (DMSO-d₆/400 MHz) 2.75-3.1 (m, 4H); 5.5 (s, 2H); 7.1-7.35 (m, 7H); 7.9 (s, 1H);
1-(3-hydroxybenzyl)-1,4,5,6-tetrahydropyrazolo[3,4-e]indazole-3-carboxamide

¹H NMR (DMSO-d₆/400 MHz) 2.65-3.05 (2t, 4H); 5.4 (s, 2H); 6.45-6.65 (m, 3H); 7.1-7.3 (m, 3H); 7.8 (s, 1H); 9.39 (s, 1H);
1-pyridin-2-yl-1,4,5,6-tetrahydropyrazolo[3,4-e]indazole-3-carboxamide

¹H NMR (DMSO-d₆/400 MHz) 2.85-3.1 (m, 4H); 7.3-7.65 (2s, 2H), 7.45 (m, 1H); 8.1 (m, 2H); 8.2 (s, 1H); 8.6 (d, 1H);
1-(3-methylphenyl)-1,4,5,6-tetrahydropyrazolo[3,4-e]indazole-3-carboxamide

¹H NMR (DMSO-d₆/400 MHz) 2.35 (s, 3H); 2.8-3.1 (m, 4H); 7.20 (s, 1H); 7.35-7.5 (m, 6H);
1-(3-chlorophenyl)-1,4,5,6-tetrahydropyrazolo[3,4-e]indazole-3-carboxamide

¹H NMR (DMSO-d₆/400 MHz) 2.8-3.1 (m, 4H); 7.2-7.35 (d+s, 3H); 7.4-7.8 (m, 4H); 1-(3-fluorophenyl)-1,4,5,6-tetrahydropyrazolo[3,4-e]indazole-3-carboxamide

¹H NMR (DMSO-d₆/400 MHz) 2.8-3.1 (2t, 4H); 7.2-7.4 (2s, 2H); 7.35 (s, 1H); 7.45-7.65 (m, 4H);
1-(6-chloropyridazin-3-yl)-1,4,5,6-tetrahydropyrazolo[3,4-e]indazole-3-carboxamide

¹H NMR (DMSO-d₆/400 MHz) 2.83 (t, 2H); 3.10 (t, 2H); 7.45 (s, 1H); 7.83 (s, 1H); 8.12 (s, 1H); 8.19 (d, 1H); 8.48 (d, 1H);
4,4-dimethyl-1-(4-methylphenyl)-1,4,5,6-tetrahydropyrazolo[3,4-e]indazole-3-carboxamide

¹H NMR (DMSO-d₆/400 MHz) 1.34 (s, 6H); 2.38 (s, 3H); 2.70 (s, 2H); 7.04 (bs, 1H); 7.24 (bs, 1H); 7.41 (dd, 4H); 7.53 (bs, 1H), 12.60 (bs, 1H).
1-pyridin-3-yl-1,4,5,6-tetrahydropyrazolo[3,4-e]indazole-3-carboxamide

¹HNMR (DMSO-d₆/400 MHz); 2.8 (t, 2H); 3.1 (t, 2H); 7.1-7.3 (d, 2H); 7.45 (s, 1H); 7.61 (m, 1H); 8.10 (d, 1H); 8.71 (d, 1H); 8.9 (s, 1H); 12.7 (bs, 1H)
1-[4-(acetylamino)phenyl]-1,4,5,6-tetrahydropyrazolo[3,4-e]indazole-3-carboxamide

¹HNMR (DMSO-d₆/400 MHz); 2.05 (s, 3H); 2.8 (t, 2H); 3.01 (t, 2H); 7.1 (s, 1H); 7.2 (s, 1H); 7.4 (s, 1H); 7.51 (d, 2H); 7.8 (d, 2H); 10.2 (s, 1H)
1-(4-aminophenyl)-1,4,5,6-tetrahydropyrazolo[3,4-e]indazole-3-carboxamide

¹HNMR (DMSO-d₆/400 MHz); 2.80 (bt, 2H); 3.04 (bt, 2H); 5.45 (bs, 2H); 6.66 (d, 2H); 7.04 (bs, 1H); 7.11 (bs, 1H); 7.16 (d, 2H); 7.32 (bs, 1H)
1-{4-[(4-methylpiperazin-1-yl)sulfonyl]phenyl}-1,4,5,6-tetrahydropyrazolo[3,4-e]indazole-3-carboxamide

¹H NMR (DMSO-d₆/400 MHz); 2.15 (m, 7H); 2.96 (m, 6H); 3.96 (bt, 2H); 7.30 (bs, 1H); 7.52 (bs, 2H); 7.93 (bs, 4H)
4-[3-(aminocarbonyl)-5,6-dihydropyrazolo[3,4-e]indazol-1 (4H)-yl]benzoic acid

¹H NMR (DMSO-d₆/400 MHz); 2.84 (t, 2H); 3.06 (t, 2H); 7.27 (bs, 1H); 7.77 (d, 2H); 8.12 (d, 2H)
1-(4-morpholin-4-ylphenyl)-1,4,5,6-tetrahydropyrazolo[3,4-e]indazole-3-carboxamide

¹H NMR (DMSO-d₆/400 MHz); 2.82 (t, 2H); 3.05 (t, 2H); 3.19 (t, 4H); 3.75 (t, 4H); 7.08 (d, 2H); 7.09 (bs, 1H); 7.16 (bs, 1H); 7.36 (bs, 1H); 7.42 (d, 2H)
1-[4-(trifluoromethoxy)phenyl]-1,4,5,6-tetrahydropyrazolo[3,4-e]indazole-3-carboxamide

¹H NMR (DMSO-d₆/400 MHz); 2.85 (bt, 2H); 3.07 (t, 2H); 7.26 (bs, 2H); 7.47 (bs, 1H); 7.59 (d, 2H); 7.78 (d, 2H); 12.70 (bs, 1H)
1-butyl-1,4,5,6-tetrahydropyrazolo[3,4-e]indazole-3-carboxamide

¹H NMR (DMSO-d₆/400 MHz); 0.86 (t, 3H); 1.27 (m, 2H); 1.73 (m, 2H); 2.76 (t, 2H); 2.97 (t, 2H); 4.20 (t, 2H); 7.05 (bs, 1H); 7.16 (bs, 1H); 7.99 (bs, 1H); 12.75 (bs, 1H)
1-(2-hydroxyethyl)-1,4,5,6-tetrahydropyrazolo[3,4-e]indazole-3-carboxamide

¹H NMR (DMSO-d₆/400 MHz); 2.72 (t, 2H); 2.98 (t, 2H); 3.67 (t, 2H); 4.05 (t, 2H); 4.80 (bs (1H); 7.08 (bs, 1H); 7.24 (bs, 1H); 7.69 (s, 1H)
1-(2,5-dimethylphenyl)-1,4,5,6-tetrahydropyrazolo[3,4-e]indazole-3-carboxamide

¹H NMR (DMSO-d 400 MHz); 1.94 (s, 3H); 2.32 (s, 3H); 2.81 (t, 2H); 3.09 (t, 2H); 6.65 (bs, 1H); 7.14 (bs, 1H); 7.20 (s, 1H); 7.29 (d, 1H); 7.33 (d, 1H); 7.39 (bs, 1H)
1-(2,2,2-trifluoroethyl)-1,4,5,6-tetrahydropyrazolo[3,4-e]indazole-3-carboxamide

¹H NMR (DMSO-d₆/400 MHz); 2.77 (t, 2H); 3.00 (t, 2H); 5.17 (q, 2H); 7.23 (bs, 2H); 8.16 (bs, 1H); 12.61 (bs, 1H)
1-(2-amino-2-oxoethyl)-1,4,5,6-tetrahydropyrazolo[3,4-e]indazole-3-carboxamide

¹H NMR (DMSO-d₆/400 MHz); 2.76 (t, 2H); 2.97 (t, 2H); 4.84 (s, 2H); 7.08-7.53 (br, 4H); 7.82 (bs, 1H); 12.69 (br, 1H)
1-[4-(1H-imidazol-2-yl)phenyl]-1,4,5,6-tetrahydropyrazolo[3,4-e]indazole-3-carboxamide

¹H NMR (DMSO-d₆/400 MHz); 2.82 (t, 2H); 3.07 (t, 2H); 7.17-7.34 (m, 5H); 7.70 (d, 2H); 8.11 (d, 2H); 12.68 (bs, 1H)
4,4-dimethyl-1-(2,2,2-trifluoroethyl)-1,4,5,6-tetrahydropyrazolo[3,4-e]indazole-3-carboxamide

¹H NMR (DMSO-d₆/400 MHz); 1.29 (s, 6H); 2.64 (s, 2H); 5.16 (q, 2H); 7.30 (bs, 1H); 7.38 (bs, 1H); 8.11 (bs, 1H); 12.63 (bs, 1H)
1-methyl-1,4,5,6-tetrahydropyrazolo[3,4-e]indazole-3-carboxamide

¹H NMR (DMSO-d₆/400 MHz); 2.79 (t, 2H); 3.01 (t, 2H); 3.95 (s, 3H); 7.09 (bs, 1H); 7.25 (bs, 1H); 8.08 (bs, 1H); 12.80 (bs, 1H)
2-(2-hydroxyethyl)-2,4,5,6-tetrahydropyrazolo[3,4-e]indazole-3-carboxamide

¹H NMR (DMSO-d₆/400 MHz); 2.77 (t, 2H); 2.85 (t, 2H); 3.07 (t, 2H); 4.30 (t, 2H); 4.97 (bs, 1H); 7.61-7.86 (br, 3H); 12.58 (bs, 1H)
8-Anilino-1-methyl-1,4,5,6-tetrahydropyrazolo[3,4-e]indazole-3-carboxamide

NMR (400 MHz, DMSO-D6) δ ppm 2.78 (m, 4H) 3.79 (s, 3H) 6.8 (in, 3H) 7.08 (m, 4H) 8.05 (s, 1H)
8-Anilino-1-(2,2,2-trifluoroethyl)-1,4,5,6-tetrahydropyrazolo[3,4-e]indazole-3-carboxamide

1H NMR (400 MHz, DMSO-D6) δ ppm 3.05 (m, 4H) 5.21 (m, 2H) 6.7 (m, 3H) 7.14 (m, 2 H) 7.26 (m, 2H) 8.07 (s, 1H)
8-amino-1,4,5,6-tetrahydropyrazolo[3,4-e]indazole-3-carboxamide

¹H NMR (400 MHz, DMSO-D6) δ ppm 2.86 (t, 2H), 3.06 (t, 2H), 7.2-7.6 (br, 4H)

### EXAMPLE 4

### 1-[4-methoxyphenyl]-1,6-dihydropyrazolo[3,4-e]indazole-3-carboxamide

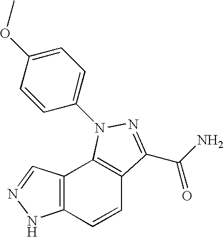

A suspension of 1-[4-methoxyphenyl]-1,4,5,6-tetrahydropyrazolo[3,4-e]indazole-3-carboxamide (137 mg, 0.44 mmols) in anhydrous dioxane (7 ml) was treated with DDQ (114 mg, 0.50 mmols) and stirred at 100° C. for 3 hours. After cooling, the mixture was evaporated to dryness, taken up with a diluted solution of K₂CO₃, filtered on buchner and washed with water to obtain 1-[4-methoxyphenyl]-1,6-dihydropyrazolo[3,4-e]indazole-3-carboxamide (98 mg, Y=73%) as a solid.

¹H NMR (DMSO-d₆/400 MHz) 3.81 (s, 3H); 7.21 (d, 2H); 7.48 (d, 1H); 7.65 (s, 1H); 7.72 (d, 2H); 8.17 (d, 1H).

By working analogously, the following compounds were prepared:
1-[4-(aminosulfonyl)phenyl]-1,6-dihydropyrazolo[3,4-e]indazole-3-carboxamide

¹H NMR (DMSO-d₆/400 MHz) 7.51 (s, 2H); 7.56 (d, 1H); 7.91 (s, 1H); 8.11 (m, 4H); 8.21 (d, 1H);
1-{4-[(methylamino)sulfonyl]phenyl}-1,6-dihydropyrazolo[3,4-e]indazole-3-carboxamide

¹H NMR (DMSO-d₆/400 MHz) 2.51 (m, 3H); 7.54 (s, 1H); 7.56 (d, 1H); 7.60 (m, 1H); 7.89-7.97 (m, 2H); 8.06-8.14 (m, 4H); 8.21 (d, 1H);
1-{4-[(butylamino)sulfonyl]phenyl)}-1,6-dihydropyrazolo[3,4-e]indazole-3-carboxamide

¹H NMR (DMSO-d₆/400 MHz) 0.81 (t, 3H); 1.30 (q, 2H); 1.38 (q, 2H); 2.83 (q, 2H); 7.11 (s, 1H); 7.54 (s, 1H); 7.56 (d, 1H); 7.73 (t, 1H); 7.80 (s, 1H); 8.10 (m, 4H); 8.21 (d, 1H);
1-{4-[(dimethylamino)sulfonyl]phenyl}-1,6-dihydropyrazolo[3,4-e]indazole-3-carboxamide

¹H NMR (DMSO-d₆/400 MHz) 2.71 (s, 6H); 7.57 (d, 1H); 8.02 (s, 1H); 8.11 (m, 4H); 8.21 (d, 1H);
1-{4-[(diprop-2-ynylamino)sulfonyl]phenyl}-1,6-dihydropyrazolo[3,4-e]indazole-3-carboxamide

¹H NMR (DMSO-d₆/400 MHz) 3.25 (s, 2H); 4.2 (d, 4H); 7.75-8.2 (2d, 2H); 7.8-8.0 (2s, 2H); 7.9 (s, 1H); 8.15 (m, 4H);
1-[4-(anilinosulfonyl)phenyl]-1,6-dihydropyrazolo[3,4-e]indazole-3-carboxamide

¹H NMR (DMSO-d₆/400 MHz) 7.1-7.21 (2m, 4H); 7.5 (s, 1H); 7.55, 8.1 (2d, 2H); 7.8, 7.9 (2s, 2H); 8.05 (s, 4H); 10.2 (s, 1H);
1-(4-{[(2-hydroxypropyl)amino]sulfonyl}phenyl)-1,6-dihydropyrazolo[3,4-e]indazole-3-carboxamide

¹H NMR (DMSO-d₆/400 MHz) 1.01 (d, 3H); 2.75 (m, 2H); 3.62 (d, 1H); 4.65 (s, 1H); 7.55 (s, 1H); 7.6-8.2 (2d, 2H); 7.75 (t, 1H); 7.9-8.0 (2s, 2H); 8.1 (s, 4H);
1-[4-(methylsulfonyl)phenyl]-1,6-dihydropyrazolo[3,4-e]indazole-3-carboxamide

¹H NMR (DMSO-d₆/400 MHz) 3.35 (s, 3H); 7.5 (s, 1H); 7.6-8.2 (2d, 2H); 7.9-8.05 (2s, 2H); 8.10-8.25 (m, 4H);
1,6-dihydropyrazolo[3,4-e]indazole-3-carboxamide

¹H NMR (DMSO-d₆/400 MHz) 7.3-7.7 (2s, 2H); 7.35-8.2 (2d, 2H); 7.45 (s, 1H);
1-phenyl-1,6-dihydropyrazolo[3,4-e]indazole-3-carboxamide

¹H NMR (DMSO-d₆/400 MHz) 7.47 (s, 1H); 7.52 (d, 1H); 7.55-7.85 (m, 7H); 8.19 (d, 1H);
1-(4-fluorophenyl)-1,6-dihydropyrazolo[3,4-e]indazole-3-carboxamide

¹H NMR (DMSO-d₆/400 MHz) 7.43-7.9 (m, 8H); 8.2 (d, 1H);
1-(4-methylphenyl)-1,6-dihydropyrazolo[3,4-e]indazole-3-carboxamide

¹H NMR (DMSO-d₆/400 MHz) 2.45 (s, 3H); 7.4-7.7 (2d, 4H); 7.5-8.2 (2d, 2H); 7.39-7.8 (2s, 2H); 7.75 (s, 1H);
1-(4-cyanophenyl)-1,6-dihydropyrazolo[3,4-e]indazole-3-carboxamide

¹H NMR (DMSO-d₆/400 MHz) 7.54 (bs, 1H); 7.55 (d, 1H); 7.93 (bs, 1H); 7.99 (bs, 1H); 8.11 (d, 2H); 8.17 (d, 2H); 8.21 (d, 1H);
1-[4-(trifluoromethyl)phenyl]-1,6-dihydropyrazolo[3,4-e]indazole-3-carboxamide

¹H NMR (DMSO-d₆/400 MHz) 7.55 (bs, 1H); 7.55 (d, 2H); 7.96 (bs, 1H); 7.95 (b's, 1H); 8.07 (d, 2H); 8.12 (d, 2H); 8.21 (d, 1H);
1-(4-chlorophenyl)-1,6-dihydropyrazolo[3,4-e]indazole-3-carboxamide

¹H NMR (DMSO-d₆/400 MHz) 7.5-8.2 (2d, ²H); 7.55-7.9 (s, 2H); 7.8 (s, 1H); 7.75-7.95 (2d, 4H);
1-(4-bromophenyl)-1,6-dihydropyrazolo[3,4-e]indazole-3-carboxamide

¹H NMR (/400 MHz) 7.47-7.53 (m, 2H); 7.81-7.90 (m, 6H); 8.19 (d, 1H); 13.64 (s, 1H);
1-(4-nitrophenyl)-1,6-dihydropyrazolo[3,4-e]indazole-3-carboxamide

¹H NMR (DMSO-d₆/400 MHz) 7.47 (s, 1H); 7.57 (d, 1H); 7.96 (bs, 1H); 8.07 (bs, 1H); 8.20 (m, 3H); 8.54 (d, 2H);
1-benzyl-1,6-dihydropyrazolo[3,4-e]indazole-3-carboxamide

¹H NMR (DMSO-d₆/400 MHz) 5.9 (s, 2H); 7.2-7.45 (m, 6H); 7.4-7.7 (2s, 2H); 8.20 (d, 1H);
1-(3-hydroxybenzyl)-1,6-dihydropyrazolo[3,4-e]indazole-3-carboxamide

¹H NMR (DMSO-d₆/400 MHz) 5.8 (s, 2H); 6.5-7.1 (m, 4H); 7.4-8.1 (2d, 2H); 7.35-7.7 (2s, 2H); 8.25 (s, 1H); 8.35 (s, 1H);
1-pyridin-2-yl-1,6-dihydropyrazolo[3,4-e]indazole-3-carboxamide

¹H NMR (DMSO-d₆/400 MHz) 7.4-7.59 (m, 2H); 7.6-8.15 (2s, 2H); 8.1 (m, 1H); 8.2-8.3 (2d, 2H); 8.8 (d, 1H); 8.9 (s, 1H);
1-(3-chlorophenyl)-1,6-dihydropyrazolo[3,4-e]indazole-3-carboxamide

¹H NMR (DMSO-d₆/400 MHz) 7.52 (bs, 1H); 7.52 (d, 1H); 7.64-7.88 (m, 3H); 7.90 (s, 1H); 7.95 (bs, 1H); 7.97 (s, 1H); 8.20 (d, 1H);
1-(3-methylphenyl)-1,6-dihydropyrazolo[3,4-e]indazole-3-carboxamide

¹H NMR (DMSO-d₆/400 MHz) 2.45 (s, 3H); 7.4-7.8 (2d, 2s, 4H); 7.45-7.65 (2s, 2H); 7.8 (s, 1H); 7.5-8.2 (2d, 2H);
1-(3-fluorophenyl)-1,6-dihydropyrazolo[3,4-e]indazole-3-carboxamide

¹H NMR (DMSO-d₆/400 MHz) 7.4-7.7 (2m, 4H); 7.5-8.2 (2d, 2H); 7.65-7.9 (2s, 2H); 7.85 (s, 1H);
1-(6-chloropyridazin-3-yl)-1,6-dihydropyrazolo[3,4-e]indazole-3-carboxamide

¹H NMR (DMSO-d₆/400 MHz) 7.45 (s, 1H); 7.6-7.65 (2s, 2H); 8.25-8.7 (2d, 2H); 8.2 (m, 2H);
1-(4-methoxyphenyl)-1,6-dihydropyrazolo[3,4-e]indazole-3-carboxylic acid

¹H NMR (DMSO-d₆/400 MHz) 3.9 (s, 3H); 7.2-7.7 (2d, 2H); 7.5-8.2 (2d, 2H); 7.69 (s, 1H);
Ethyl 1-phenyl-1,6-dihydropyrazolo[3,4-e]indazole-3-carboxylate

¹H NMR (CDCl₃/400 MHz) 1.51 (t, 3M); 4.57 (q, 2H); 7.51 (d, 1H); 7.72 (m, 5H); 7.82 (s, 1H); 8.29 (d, 1H);
Ethyl 1-(4-methoxyphenyl)-1,6-dihydropyrazolo[3,4-e]indazole-3-carboxylate

¹H NMR (DMSO-d₆/400 MHz) 1.4 (t, 3H); 3.9 (s, 3H); 4.45 (q, 2H); 7.25-7.7 (2d, 2H); 7.55-8.2 (2d, 2H); 7.6 (s, 1H);
N-methyl-1-[4-(aminosulfonyl)phenyl]-1,6-dihydropyrazolo[3,4-e]indazole-3-carboxamide

¹H NMR (DMSO-d₆/400 MHz) 2.85 (d, 3H), 7.45 (s, 2H); 7.6-8.2 (2d, 2H); 7.9 (s, 1H); 8.10 (m, 4H); 8.5 (d, 1H); 13.73 (s, 1H);
N-methyl-1-{4-[(butylamino)sulfonyl]phenyl}-1,6-dihydropyrazolo[3,4-e]indazole-3-carboxamide

¹H NMR (DMSO-d₆/400 MHz) 0.8 (t, 3H); 1.2-1.4 (2m, 4H); 2.81 (m, 5H); 7.45-8.2 (2d, 2H); 7.75 (t, 1H); 7.95 (s, 1H); 8.10 (m, 4H); 8.5 (m, 1H); 13.7 (s, 1H);
N-methyl-1-{4-[(dimethylamino)sulfonyl]phenyl}-1,6-dihydropyrazolo[3,4-e]indazole-3-carboxamide

¹H NMR (DMSO-d₆/400 MHz) 2.7 (s, 6H); 2.85 (d, 3H); 7.6-8.2 (2d, 2H); 8.05 (s, 1H); 8.07-8.15 (2d, 4H);
N-methyl-1-[4-(methylsulfonyl)phenyl]-1,6-dihydropyrazolo[3,4-e]indazole-3-carboxamide

¹H NMR (DMSO-d₆/400 MHz) 2.83 (d, 3H); 3.18 (s, 3H); 7.6-8.25 (2d, 2H); 8.05 (s, 1H); 8.15-8.20 (m, 4H); 8.45 (m, 1H);
N-(allyloxy)-1-{4-[(butylamino)sulfonyl]phenyl}-1,6-dihydropyrazolo[3,4-e]indazole-3-carboxamide

¹H NMR (DMSO-d₆/400 MHz) 0.8 (t, 3H); 1.2-1.4 (2m, 4H); 2.8 (q, 2H); 4.45 (d, 2H); 5.2-5.4 (2d, 2H); 6.1 (m, 1H); 7.55-8.15 (2d, 2H); 7.7 (t, 1H); 7.9 (s, 1H); 8.1 (s, 4H);
7,8,9,10-tetrahydro[1,4]diazepino[1,2-b]pyrazolo[3,4-g]indazol-6(3H)-one

¹H NMR (DMSO-d₆/400 MHz) 2.24 (m, 2H); 3.21 (m, 2H); 4.68 (bt, 2H); 7.33-7.38 (dd, 1H); 7.73-7.79 (dd, 1H); 8.21 (s, 1H); 8.32 (bs, 1H); 13.36 (bs, 1H).
1-pyridin-3-yl-1,6-dihydropyrazolo[3,4-e]indazole-3-carboxamide

¹HNMR (DMSO-d₆/400 MHz); 7.45 (d, 2H); 7.75 (m, 2H); 7.91 (s, 1H); 8.2 (d, 1H); 8.35 (d, 1H); 8.81 (d, 1H); 9.1 (s, 1H)
1-[4-(acetylamino)phenyl]-1,6-dihydropyrazolo[3,4-e]indazole-3-carboxamide

¹H NMR (DMSO-d₆/400 MHz); 2.10 (s, 3H); 7.5 (bs, 1H); 7.48 (d, 1H); 7.73 (d, 2H); 7.79 (bs, 1H); 7.87 (d, 2H); 8.17 (d, 1H)
4-[3-(aminocarbonyl)pyrazolo[3,4-e]indazol-1 (6H)-yl]benzoic acid

¹H NMR (DMSO-d₆/400 MHz); 7.53 (bs, 1H); 7.54 (d, 1H); 7.89 (bs, 1H); 7.95 (bs, 1H); 8.01 (d, 2H); 8.20 (d, 1H); 8.24 (d, 2H)
1-{4-[(4-methylpiperazin-1-yl)sulfonyl]phenyl}-1,6-dihydropyrazolo[3,4-e]indazole-3-carboxamide

¹H NMR (DMSO-d₆/400 MHz); 2.15 (s, 3H); 2.39 (bt, 4H); 3.01 (bt, 4H); 7.55 (d, 1H); 7.56 (bs, 1H); 7.91n(bs, 1H); 8.02 (bs, 1H); 8.04 (d, 2H); 8.16 (d, 2H); 8.21 (d, 1H); 13.72 (bs, 1H)
1-[4-(trifluoromethoxy)phenyl]-1,6-dihydropyrazolo[3,4-e]indazole-3-carboxamide

¹H NM (DMSO-d₆/400 MHz); 7.52 (bs, 1H); 7.53 (d, 1H); 7.70 (d, 2H); 7.86 (bs, 2H); 8.01 (d, 2H); 8.20 (d, 1H); 13.69 (bs, 1H)
4-[3-(ethoxycarbonyl)pyrazolo[3,4-e]indazol-1 (6H)-yl]benzoic acid

¹H NMR (DMSO-d₆/400 MHz); 1.39 (t, 3H); 4.43 (q, 2H); 7.62 (d, 1H); 7.93 (s, 1H); 7.98 (d, 2H); 8.09 (d, 2H); 8.24 (d, 2H); 13.27 (bs, 1H); 13.43 (bs, 1H)
1-(4-morpholin-4-ylphenyl)-1,6-dihydropyrazolo[3,4-e]indazole-3-carboxamide

¹H NMR (DMSO-d₆/400 MHz); 3.26 (bt, 4H); 3.78 (bt, 4H); 7.19 (d, 2H); 7.42 (bs, 1H); 7.46 (d, 1H); 7.63 (d, 2H); 7.71 (bs, 1H); 7.75 (bs, 1H); 8.16 (d, 1H); 13.59 (bs, 1H)
1-(2-hydroxyethyl)-1,6-dihydropyrazolo[3,4-e]indazole-3-carboxamide

¹H NMR (DMSO-d₆/400 MHz); 3.92 (t, 2H); 4.67 (t, 2H); 7.31 (bs, 1H); 7.38 (d, 1H); 7.61 (bs, 1H); 8.06 (d, 1H); 8.47 (bs, 1H); 13.50 (bs, 1H)
1-(2,5-dimethylphenyl)-1,6-dihydropyrazolo[3,4-e]indazole-3-carboxamide

¹H NMR (DMSO-d₆/400 MHz); 1.94 (s, 3H); 2.37 (s, 3H); 7.20 (s, 1H); 7.40 (m, 4H); 7.47 (d, 1H); 7.79 (bs, 1H); 8.18 (d, 1H); 13.58 (bs, 1H)
1-(2-aminoethyl)-1,6-dihydropyrazolo[3,4-e]indazole-3-carboxamide hydrochloride

¹H NMR (DMSO-d₆/400 MHz); 3.38 (t, 2H); 4.90 (t, 2H); 7.43 (d, 1H); 7.44 (bs, 1H); 7.89 (bs, 1H); 8.08 (d, 1H); 8.21 (bs 3H); 8.60 (s, 1H)
1-(2,2,2-trifluoroethyl)-1,6-dihydropyrazolo[3,4-e]indazole-3-carboxamide

¹H NMR (DMSO-d₆/400 MHz); 5.63 (m, 2H); 7.46 (d, 1H); 7.50 (bs, 1H); 8.11 (d, 1H); 8.62 (s, 1H); 13.62 (bs, 1H)
1-[4-(1H-imidazol-2-yl)phenyl]-1,6-dihydropyrazolo[3,4-e]indazole-3-carboxamide

¹H NMR (DMSO-d₆/400 MHz); 7.25 (s, 2H); 7.50 (bs, 1H); 7.52 (d, 1H); 7.86 (bs, 2H); 7.94 (d, 2H); 8.20 (d, 1H); 8.23 (d, 2H) 12.50 (bs, 1H)
1-methyl-1,6-dihydropyrazolo[3,4-e]indazole-3-carboxamide

¹H NMR (DMSO-d₆/400 MHz); 4.35 (s, 3H); 7.35 (bs, 1H); 7.42 (d, 1H); 7.65 (bs, 1H); 8.10 (d, 1H); 8.56 (s, 1H); 13.56 (bs, 1H)
8,9-dihydro-3H-pyrazino[1,2-b]pyrazolo[3,4-g]indazol-6(7H)-one

¹H NMR (DMSO-d₆/400 MHz); 3.72 (m, 2H); 4.57 (t, 2H); 7.43 (d, 1H); 7.83 (d, 1H); 8.27 (bs, 2H); 13.42 (bs, 1H)
2-(2-aminoethyl)-2,6-dihydropyrazolo[3,4-e]indazole-3-carboxamide hydrochloride

¹H NMR (DMSO-d₆/400 MHz); 3.55 (t, 2H); 4.84 (t, 2H); 7.42 (d, 1H); 7.66 (d, 1H); 8.00-8.12 (bs, 6H); 8.27 (s, 1H)
2-(2-hydroxyethyl)-2,6-dihydropyrazolo[3,4-e]indazole-3-carboxamide

¹H NMR (DMSO-d₆/400 MHz); 3.84 (t, 2H); 4.67 (t, 2H); 7.35 (d, 1H); 7.62 (d, 1H); 7.90 (bs, 1H); 8.177 (bs, 1H); 8.25 (s, 1H)
2-methyl-2,6-dihydropyrazolo[3,4-e]indazole-3-carboxamide

¹H NMR (DMSO-d₆/400 MHz); 4.27 (s, 3H); 7.40 (d, 1H); 7.66 (d, 1H); 7.93 (bs, 1H); 8.04 (bs, 1H); 8.25 (s, 1H) 13.38 (bs, 1H)
1-anilino-8,9-dihydro-3H-pyrazino[1,2-b]pyrazolo[3,4-g]indazol-6(7H)-one

NMR (400 MHz, DMSO-D6) δ ppm 3.74 (m, 2H) 4.64 (m, 2H) 6.82 (t, J=7.32 Hz, 1H) 7.25 (t, J=7.32 Hz, 2H) 7.36 (d, J=9.02 Hz, 1H) 7.57 (d; J=8.5 Hz, 2H) 7.84 (bs, 1H) 7.88 (d, J=9.02 Hz, 1H) 8.34 (bs, 1H)

### EXAMPLE 5

### 1-(4-methoxy-phenyl)-1,6-dihydropyrazolo[3,4-e]indazol-3-amine

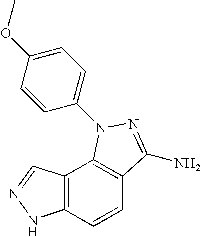

A solution of 1-[4-methoxyphenyl]-1,6-dihydropyrazolo[3,4-e]indazole-3-carboxamide (100 mg; 0.325 mmols) in 2.5 N NaOH (500 mg in 5 ml of water; 12.5 mmols) was treated with a 1M solution of NaClO (0.325 ml). The resulting mixture was heated at 100° C. for 15 minutes. After cooling to room temperature, the solution was filtered and neutralized with HCl. The resulting precipitate was extracted with ethyl acetate, dried over Na₂SO₄ and evaporated to dryness. The crude material was then chromatographed on silica gel eluted with ethyl acetate to obtain 1-(4-methoxy-phenyl)-1,6-dihydropyrazolo[3,4-e]indazol-3-amine (45 mg; Y=49%) as a brown solid.

¹H NMR (DMSO-d₆/400 MHz) 5.65 (bs, 2H); 7.11 (d, 2H); 7.17 (d, 1H); 7.56 (d, 2H); 7.67 (d, 1H); 7.80 (s, 1H).

### EXAMPLE 6

### 1-[1-(4-methylphenyl)-1,6-dihydropyrazolo[3,4-e]indazol-3-yl] ethanone

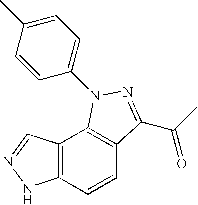

Triethylamine (0.82 ml; 6 mmols) was added to a 3M solution of EtMgCl in THF (0.67 ml; 2 mmols) at 0° C. under argon atmosphere. After 10 min, a solution of ethyl 1-(4-methylphenyl)-7-trityl-1,7-dihydropyrazolo[3,4-e]indazole-3-carboxylate (564 mg; 1 mmol) in anhydrous THY (6 ml) was added dropwise. After leaving at 0° C. for 1 hour and 30 min at room temperature the resulting mixture was poured into a 20% NaH₂PO₃ solution and extracted with ethyl acetate. The organic extracts were collected, dried over Na₂SO₄ and evaporated to dryness. The crude material was chromatographed on silica gel eluted with hexane/ethyl acetate 8/2 to obtain 1-[1-(4-methylphenyl)-7-trityl-1,7-dihydropyrazolo[3,4-e]indazol-3-yl]ethanone (120 mg; Y=22%) as a white solid. The latter was then suspended in acetone (6 ml) and treated with few drops of 37% HCl. The resulting mixture was left at room temperature for 1.5 hours and filtered to give 1-[1-(4-methylphenyl)-1,6-dihydropyrazolo[3,4-e]indazol-3-yl]ethanone (50 mg; Y=78%) as a white solid.

¹H NMR (DMSO-d₆/400 MHz) 2.41 (s, 3H); 2.84 (t, 2H); 3.06 (t, 2H); 7.13 (s, 1H); 7.40 (d, 2H); 7.50 (d, 2H).

### EXAMPLE 7

### 1-(4-methoxyphenyl)-1,6-dihydropyrazolo[3,4-e]indazole-3-carbonitrile

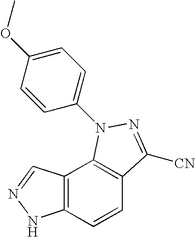

A suspension of 1-[4-methoxyphenyl]-1,6-dihydropyrazolo[3,4-e]indazole-3-carboxamide (20 mg; 0.065 mmols) in anhydrous THF (0.5 ml) was treated at room temperature under argon atmosphere with pyrridine (0.05 ml; 0.65 mmols) and trifluoroacetic anhydride (0.05 ml; 0.39 mmols). The resulting solution was left at room temperature for 1 hour, diluted with water and filtered to obtain 1-(4-methoxyphenyl)-1,6-dihydropyrazolo[3,4-e]indazole-3-carbonitrile (16 mg; Y=885%) as a white solid

¹H NMR (DMSO-d₆/400 MHz) 3.89 (s, 3H); 7.23 (d, 2H); 7.65 (d, 1H); 7.74 (d, 2H), 7.75 (s, 1H), 7.78 (db, 1H).

### EXAMPLE 8

### 1-(4-methoxyphenyl)-1,6-dihydropyrazolo[3,4-e]indazole-3-carbohydrazide

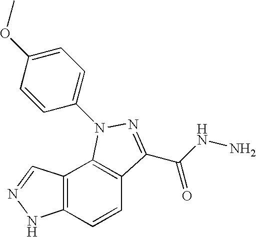

Ethyl 1-(4-methoxyphenyl)-1,6-dihydropyrazolo[3,4-e]indazole-3-carboxylate (100 mg; 0.295 mmols) was suspended on MeOH (5 ml) and treated with hydrazine hydrate (2.5 ml). The mixture was refluxed for 7 hours, after cooling, concentrated under reduced pressure, diluted with water and filtred to obtain 1-(4-methoxyphenyl)-1,6-dihydropyrazolo[3,4-e]indazole-3-carbohydrazide (85 mg; Y=89%) as a white solid

¹H NMR (DMSO-d₆/400 MHz) 3.88 (s, 3H); 4.51 (bs, 2H); 7.22 (d, 2H); 7.47 (s, 1H); 7.49 (d, 1H); 7.72 (d, 2H); 8.13 (d, 1H).

By working analogously, the following compound was prepared:
1-(4-methoxyphenyl)-1,4,5,6-tetrahydropyrazolo[3,4-e]indazole-3-carbohydrazide

¹H NMR (DMSO-d₆/400 MHz) 2.80 (m, 2H); 3.05 (m, 2H); 3.82 (s, 3H); 4.38 (bs, 2H); 7.10 (d, 2H); 7.19 (bs, 1H); 7.50 (d, 2H).

### EXAMPLE 9

### N′-hydroxy-1-(4-methoxyphenyl)-1,6-dihydropyrazolo[3,4-e]indazole-3-carboximidamide

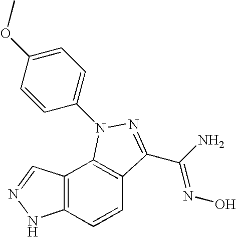

1-(4-methoxyphenyl)-1,6-dihydropyrazolo[3,4-e]indazole-3-carbonitrile (10 mg; 0.035 mmols) was suspended in ethanol (2.5 ml) and treated with hydroxylamine hydrochloride (116 mg; 1.68 mmols) and with a solution of sodium carbonate (146 mg) in water (1 ml). The resulting mixture was refluxed for 4 hours, after cooling, concentrated under vacuum, diluted with water and filtered to give N′-hydroxy-1-(4-methoxyphenyl)-1,6-dihydropyrazolo[3,4-e]indazole-3-carboximidamide (8.3 mg; Y=76%) as a white solid.

¹H NMR (DMSO-d₆/400 MHz) 3.9 (s, 3H); 7.2-7.71 (2d, 4H); 7.69 (s, 1H); 7.45-8.15 (2d, 2H); 9.1 (s, 1H).

### EXAMPLE 10

### 1-(4-methoxyphenyl)-1,6-dihydropyrazolo[3,4-e]indazole-3-carboxylic acid

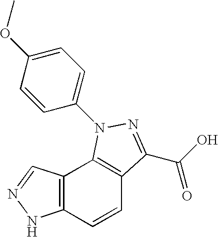

To a suspension of ethyl 1-(4-methoxyphenyl)-1,6-dihydropyrazolo[3,4-e]indazole-3-carboxylate (400 mg; 1.18 mmols) in methanol (10 ml) N NaOH (5.9 ml) was added dropwise. The resulting mixture was kept at 80° C. for 2 hours. After cooling, the solution was acidified with 2N HCl and the product was filtered on buckner to give 1-(4-methoxyphenyl)-1,6-dihydropyrazolo[3,4-e]indazole-3-carboxylic acid (360 mg; Y=98%).

¹H NMR (DMSO-d₆/400 MHz) 2.85-3.15 (2t, 4H); 3.85 (s, 3H); 7.1 (s, 1H); 7.15-7.45 (2d, 4H).

By working analogously, the following compounds were prepared:
1-(4-bromophenyl)-1,4,5,6-tetrahydropyrazolo[3,4-e]indazole-3-carboxylic acid

¹H NMR (DMSO-d₆/400 MHz) 2.8-3.1 (m, 4H); 7.95 (s, 1H); 7.45-7.75 (d, 4H);
1-{4-[(butylamino)sulfonyl]phenyl}-1,4,5,6-tetrahydropyrazolo[3,4-e]indazole-3-carboxylic acid

¹H NMR (DMSO-d₆/400 MHz) 0.8 (t, 3H); 1.2-1.4 (m, 4H); 2.8 (dd, 2H); 2.85-3.15 (2t, 4H); 7.35 (s, 1H); 7.7 (t, 1H); 7.85-8.05 (2d, 4H);
4,4-dimethyl-1-(4-methylphenyl)-1,4,5,6-tetrahydropyrazolo[3,4-e]indazole-3-carboxylic acid

¹H NMR (DMSO-d₆/400 MHz) 1.34 (s, 6H); 2.39 (s, 3H); 2.71 (s, 2H); 7.03 (bs, 1H); 7.42 (dd, 4H); 12.70 (bs, 2H).

### EXAMPLE 11

### N-hydroxy-1-(4-methoxyphenyl)-1,6-dihydropyrazolo[3,4-e]indazole-3-carboxamide

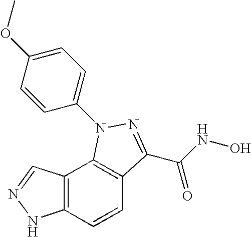

To a solution of 1-(4-methoxyphenyl)-1,6-dihydropyrazolo[3,4-e]indazole-3-carboxylic acid (100 mg; 0.325 mmols) in DMF (2 ml), carbonyl diimidazole (106 mg; 0.65 mmols) was added and the mixture was stirred for 1 hour. Na₂CO₃ (65 mg; 0.60 mmols) and hydroxylamine hydrochloride (45 mg; 0.65 mmols) were then added and the mixture was stirred for 3 hours at room temperature. After evaporation of the solvent under reduced pressure, the crude material was taken up with water and filtered on buckner to give a solid compound that was further purified by chromatography on silica gel eluted with methylene chloride/methanol 10/1, to afford the desired N-hydroxy-1-(4-methoxyphenyl)-1,6-dihydropyrazolo[3,4-e]indazole-3-carboxamide (50 mg; Y=48%).

¹H NMR (DMSO-d₆/400 MHz) 2.8-3.1 (m, 4H); 3.91 (s, 3H); 7.2 (s, 1H); 7.15-7.45 (2d, 4H); 8.85 (s, 1H); 10.81 (s, 1H).

By working analogously, the following compounds were prepared:
N-(allyloxy)-1-{4-[(butylamino)sulfonyl]phenyl}-1,4,5,6-tetrahydropyrazolo[3,4-e]indazole-3-carboxamide

¹H NMR (DMSO-d₆/400 MHz) 0.8 (m, 3H); 1.21-1.39 (m, 4H); 2.75 (m, 2H); 2.85-3.1 (m, 4H); 4.39 (d, 2H); 5.23-5.39 (2d, 2H); 5.9 (m, 1H); 7.4 (s, 1H); 7.65 (t, 1H); 7.85-8.0 (2d, 4H); 11.45 (s, 1H);
N-(allyloxy)-1-(4-methoxyphenyl)-1,4,5,6-tetrahydropyrazolo[3,4-e]indazole-3-carboxamide

¹H NMR (DMSO-d₆/400 MHz) 2.85-3.15 (m, 4H); 3.8 (s, 3H); 4.4 (d, 2H); 5.2-5.35 (2d, 2H); 5.9 (m, 1H); 7.1 (s, 1H); 7.15-7.45 (2d, 4H); 11.39 (s, 1H).

### EXAMPLE 12

### N-methyl-1-[4-(methylsulfonyl)phenyl]-1,4,5,6-tetrahydropyrazolo[3,4-e]indazole-3-carboxamide

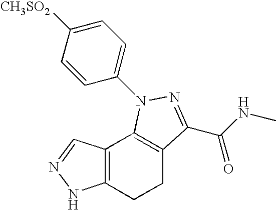

Ethyl 1-[4-(methylsulfonyl)phenyl]-1,4,5,6-tetrahydropyrazolo[3,4-e]indazole-3-carboxylate (200 mg; 0.52 mmols) was dissolved in a 33% solution of methylamine in ethanol (10 ml) and stirred at 65° C. overnight. After evaporation of the solvent under reduced pressure, the residue was purified by chroatography on silica gel eluted with methylene chloride/methanol 10/1, to give N-methyl-1-[4-(methylsulfonyl)phenyl]-1,4,5,6-tetrahydropyrazolo[3,4-e]indazole-3-carboxamide (180 mg; Y=93%).

¹H NMR (DMSO-d₆/400 MHz) 2.75 (d, 3H); 2.85-3.1 (2m, 4H); 3.25 (s, 3H); 7.45 (s, 1H); 7.95-8.18 (2d, 4H).

By working analogously, the following compounds were prepared:
1-[4-(aminosulfonyl)phenyl]-N-methyl-1,4,5,6-tetrahydropyrazolo[3,4-e]indazole-3-carboxamide

¹H NMR (DMSO-d₆/400 MHz) 2.75 (d, 3H); 2.8-3.1 (m, 4H); 7.35 (s, 1H); 7.45 (s, 2H); 7.85-8.05 (2d, 4H); 8.15 (d, 1H);
1-{4-[(butylamino)sulfonyl]phenyl}-N-methyl-1,4,5,6-tetrahydropyrazolo[3,4-e]indazole-3-carboxamide

¹H NMR (DMSO-d₆/400 MHz) 0.8 (q, 3H); 1.2-1.4 (2m, 4H); 2.75 (d, 3H); 2.8 (m, 2H); 2.85-3.1 (2m, 4H); 7.35 (s, 1H); 7.65 (t, 1H); 7.85-7.95 (2d, 4H); 8.15 (q, 1H);
1-{4-[(dimethylamino)sulfonyl]phenyl}-N-methyl-1,4,5,6-tetrahydropyrazolo[3,4-e]indazole-3-carboxamide

¹H NMR (DMSO-d₆/400 MHz) 2.65 (s, 6H); 2.85-3.1 (2m, 4H); 7.45 (s, 1H); 7.95 (m, 4H); 8.18 (m, 1H).

### EXAMPLE 13

### Ethyl 2-(3-aminopropyl)-2,4,5,6-tetrahydropyrazolo[3,4-e]indazole-3-carboxylate hydrochloride

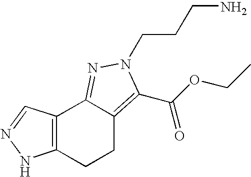

A solution of ethyl 7-trityl-1,4,5,7-tetrahydropyrazolo[3,4-e]indazole-3-carboxylate (2 g; 4.2 mmols) in DMF (20 ml) was cooled to 0° C. and treated dropwise with 1M lithium t-butoxide in THF (8.4 ml; 8.4 mmols). The solution was kept at OC for 30 min and boc-aminopropyl bromide (1.1 g; 4.6 mmols) in TIE (5 ml) was added dropwise. The mixture was stirred overnight at room temperature, poured in NaHPO4 aqueous solution and extracted with ethyl acetate. The organic layer was evaporated to dryness and the crude material dissolved in dioxane (20 ml), treated with 37% hydrochloric acid (8 ml) and stirred at room temperature for four hours. After removing the solvent under reduced pressure, the residue was taken up with ethyl acetate and filtered to give ethyl 2-(3-aminopropyl)-2,4,5,6-tetrahydropyrazolo[3,4-e]indazole-3-carboxylate hydrochloride (1.21 g; Y=88%).

¹H NMR (DMSO-d₆/400 MHz) 1.33 (t, 3H); 2.55 (m, 2H); 2.76 (m, 2H); 2.83 (t, 2H); 3.01 (t, 2H); 4.31 (q, 2H); 4.49 (t, 2H); 7.79 (s, 1H).

### EXAMPLE 14

### 4,5,7,8,9,10-hexahydro[1,4] diazepino[1,2-b]pyrazolo[3,4-g]indazol-6(3H)-one

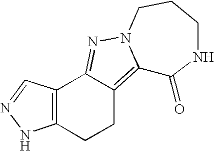

A solution of ethyl 2-(3-aminopropyl)-2,4,5,6-tetrahydropyrazolo[3,4-e]indazole-3-carboxylate (1.21 g; 3.71 mmols) in methanol (50 ml) was treated with cesium carbonate (2.42 g; 7.43 mmols) and stirred at room temperature for one day. The solution was evaporated to dryness, taken up with water and, after vigorous stirring, filtred to give 4,5,7,8,9,10-hexahydro[1,4]diazepino[1,2-b]pyrazolo[3,4-g]indazol-6(3H)-one (0.715 g; Y=79%).

¹H NMR (DMSO-d₆/400 MHz) 2.05 (in, 2H); 2.75 (m, 2H); 2.86 (m, 2H), 3.14 (m, 2H), 4.30 (t, 2H); 7.79 (bs, 1H); 8.09 (bs, 1H).

By working analogously, the following compounds were prepared:
5,5-dimethyl-4,5,7,8,9,10-hexahydro[1,4]diazepino[1,2-b]pyrazolo[3,4-g]indazol-6(3H)-one

¹H NMR (DMSO-d₆/400 MHz) 1.24 (s, 6H); 1.99 (m, 2H); 2.62 (s, 2H); 3.04 (bt, 2H); 4.25 (t, 2H); 7.72 (s, 1H); 8.29 (bs, 1H); 12.54 (bs, 1H);
5,5-dimethyl-4,5,8,9-tetrahydro-3H-pyrazino[1,2-b]pyrazolo[3,4-g]indazol-6(7H)-one

¹H NMR (DMSO-d₆/400 MHz) 1.35 (s, 6H); 2.64 (bs, 2H); 3.54 (t, 2H); 4.20 (t, 2H); 7.81 (bs, 1H); 8.14 (bs, 1H); 12.57 (bs, 1H).
4,5,8,9-tetrahydro-3H-pyrazino[1,2-b]pyrazolo[3,4-g]indazol-6(7H)-one

¹HNMR (DMSO-d₆/400 MHz); 2.65 (t, 2H); 2.95 (t, 2H); 3.56 (m, 2H); 4.19 (t, 2H); 7.8 (bs, 1H); 8.05 (s, 1H); 12.7 (bs, 1H)
1-anilino-4,5,8,9-tetrahydro-3H-pyrazino[1,2-b]pyrazolo[3,4-g]indazol-6(7H)-one

NMR (400 MHz, DMSO-D6) 8 ppm 2.85 (m, 2H) 3.03 (m, 2H) 3.60 (m, 2H) 4.25 (m, 2H) 6.75 (m, 1H) 17.19 (m, 2H) 7.30 (bs, 1H) 7.45 (m, 2H) 8.12 (s, 1H)

1. A method for treating diseases caused by and/or associated with an altered protein kinase activity which comprises administering to a mammal in need thereof an effective amount of a compound of formula (I) 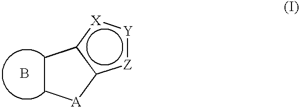
wherein
X, Y and Z, being part of an aromatic ring are selected, each independently, from the group consisting of N, NR₁, S, O and CR₁;
R₁ is selected from the group consisting of hydrido, lower alkyl, perfluorinated lower alkyl, heterocyclyl, CN, CO₂R′, COCF₃, COR′, CONR′R″, NR′R″, C(═NR′)NR′R″, CONHNH₂, CONHOR′, NHCOR′, CH₂NH₂, and CH₂NHCOR′; or R₁ may form, when part of Z or Y, a 5 to 7 membered ring together with the remaining of Y or Z, as per the formulae below 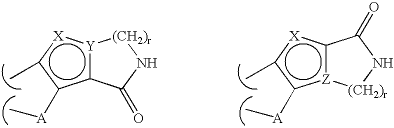
R′ and R″ are selected, each independently, from the group consisting of hydrido, hydroxy, alkyl, hydroxyalkyl, alkenyl, alkynyl, aryl, arylalkyl, heterocyclyl or heterocyclyl-alkyl;
B is an aromatic 5 or 6 membered ring having from 0 to 3 heteroatoms selected from S, O and N;
A is selected from the group consisting of —(CH₂)ₘ—,
—(CH₂)ₙ—CH═CH—(CH₂)ₙ— and —(CR_{z}R_{y})ₚ—;
R_{z} and R_{y} are selected, each independently, from hydrido or lower alkyl;
each of the X,Y,Z and B rings being optionally further substituted by one or more -L-R₂ groups, wherein
L represents, each independently, a single bond, an alkylidene group or a divalent group selected from NH, NHCO, CONH, NHCONH, SO₂NH and NHSO₂;
R₂ is, each independently, hydrido, alkyl, 5 to 12 membered mono- or bi-cyclic ring having from 0 to 3 heteroatoms selected from S, O and N, optionally substituted with one or more —(CH₂)_{q}—R₃ groups; or R₂ is a group of formula 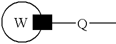
W is a 3 to 7 membered ring having one N heteroatom directly linked to Q and from 0 to 2 additional heteroatoms selected from the group consisting of S, SO, SO₂, O, N and NR′, wherein R′ is as above defined;
Q is a divalent group selected from CO, SO₂ and (CH₂)ₙ;
R₃ is selected, each independently, from the group consisting of alkyl halogen, CF₃, OCF₃, NO₂, CN, C(═NR′)NR′R″, OR′, SR′, OCOR′, OCONR′R″, COCF₃, COR′, CO₂R′, CONR′R″, SO₂R′, SO₂NR′R″, NR′R″, NR′COR′, NR′COOR′, NR′CONR′R″, NR′SO₂R′, NR′SO₂NR′R″, wherein R′ and R″ are as above defined;
m is an integer from 1 to 4;
n is, each independently, 0, 1, or 2;
p is 1 or 2;
q is, each independently, 0 or an integer from 1 to 3;
r is an integer from 1 to 3;
or isomers, tautomers, carriers, prodrugs, and pharmaceutically acceptable salts thereof.

2. The method of claim 1 wherein the disease caused by and/or associated with an altered protein kinase activity is a cell proliferative disorder selected from the group consisting of cancer, Alzheimer's disease, viral infections, auto-immune diseases and neurodegenerative disorders.

3. The method of claim 2 wherein the cancer is selected from carcinoma, squamous cell carcinoma, hematopoietic tumors of lymphoid or myeloid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer and Kaposi's sarcoma.

4. The method of claim 1 wherein the cell proliferative disorder is selected from benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

5. The method of claim 1 which provides tumor angiogenesis and metastasis inhibition.

6. The method of claim 1 further comprising subjecting the mammal in need thereof to a radiation therapy or chemotherapy regimen in combination with at least one cytostatic or cytotoxic agent.

7. The method of claim 1 wherein the mammal in need thereof is a human.

8. The method of claim 1 which comprises administering to a mammal in need thereof an effective amount of a compound of formula (Ic) 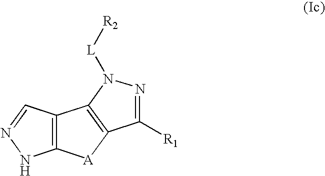
wherein R₁, L and R₂ are, each independently, as defined in claim 1, and A is selected from the group consisting of —CH₂—, —CH₂—CH₂—, —CH═CH— and —CH₂—C(CH₃)₂—.

9. The method of claim 1 which comprises administering to a mammal in need thereof an effective amount of a compound of formula (Id) 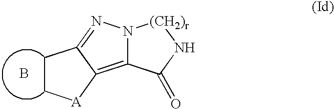
wherein r and B are as defined in claim 1, A is selected from the group consisting of —CH₂—, —CH₂—CH₂—, —CH═CH— and —CH₂—C(CH₃)₂—, and the B ring being optionally further substituted as defined in claim 1.

10. The method of claim 1 which comprises administering to a mammal in need thereof an effective amount of a compound of formula (Ie) or (If) 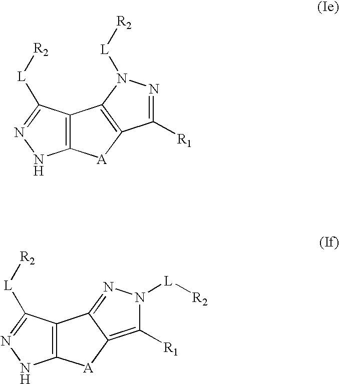
wherein L and R₂ are, each independently and the same or different in each occasion, as defined in claim 1; A is selected from the group consisting of —CH₂—CH₂—, —CH═CH— and —CH₂—C(CH₃)₂—; and R₁ is a group selected from NR′R″, CN, CO₂R′, COR′, CONR′R″, CONHOR′, CONHNH₂ and C(═NOH)NR′R″, wherein R′ and R″ are, the same or different, hydrido or lower alkyl.

11. The method of claim 1 which comprises administering to a mammal in need thereof an effective amount of a compound of formula (Ig) 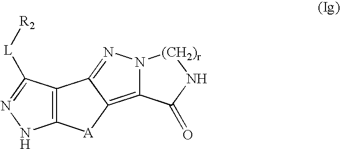
wherein L, R₂ and r are as defined in claim 1 and A is selected from the group consisting of —CH₂—CH₂—, —CH═CH— and —CH₂—C(CH₃)₂—.

12. A method for inhibiting protein kinase activity which comprises contacting the said kinase with an effective amount of a compound of formula (I) as defined in claim 1.

13. A compound represented by formula (I) 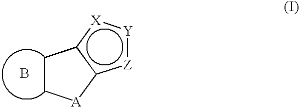
wherein
X, Y and Z, being part of an aromatic ring are selected, each independently, from the group consisting of N, NR₁, S, O and CR₁;
R₁ is selected from the group consisting of hydrido, lower alkyl, perfluorinated lower alkyl, heterocyclyl, CN, CO₂R′, COCF₃, COR′, CONR′R″, NR′R″, C(═NR′)NR′R″, CONHNH₂, CONHOR′, NHCOR′, CH₂NH₂, and CH₂NHCOR′; or R₁ may form, when part of Z or Y, a 5 to 7 membered ring together with the remaining of Y or Z, as per the formulae below 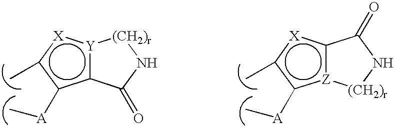
R′ and R″ are selected, each independently, from the group consisting of hydrido, hydroxy, alkyl, hydroxyalkyl, alkenyl, alkynyl, aryl, arylalkyl, heterocyclyl or heterocyclyl-alkyl;
B is an aromatic 5 or 6 membered ling having from 0 to 3 heteroatoms selected from S, O and N;
A is selected from the group consisting of —(CH₂)ₘ—,
—(CH₂)ᵣ—CH═CH—(CH₂)ₙ— and —(CR_{z}R_{y})ₚ—;
R_{z} and R_{y} are selected, each independently, from hydrido or lower alkyl;
each of the X,Y,Z and B rings being optionally further substituted by one or more -L-R₂ groups, wherein
L represents, each independently, a single bond, an alkylidene group or a divalent group selected from NH NHCO, CONH, NHCONH, SO₂NH and NHSO₂;
R₂ is, each independently, hydrido, alkyl, 5 to 12 membered mono- or bi-cyclic ring having from 0 to 3 heteroatoms selected from S, O and N, optionally substituted with one or more —(CH₂)_{q}—R₃ groups; or R₂ is a group of formula 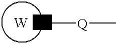
W is a 3 to 7 membered ring having one N heteroatom directly linked to Q and from 0 to 2 additional heteroatoms selected from the group consisting of S, SO, SO₂, O, N and NR′, wherein R′ is as above defined;
Q is a divalent group selected from CO, SO₂ and (CH₂)ₙ;
R₃ is selected, each independently, from the group consisting of alky, halogen, CF₃, OCF₃, NO₂, CN, C(═NR′)NR′R″, OR′, SR′, OCOR′, OCONR′R″, COCF₃, COR′, CO₂R′, CONR′R″, SO₂R′, SO₂NR′R″, NR′R″, NR′COR′, NR′COOR′, NR′CONR′R″, NR′SO₂R′, NR′SO₂NR′R″, wherein R′ and R″ are as above defined;
m is an integer from 1 to 4;
n is, each independently, 0, 1, or 2;
p is 1 or 2;
q is, each independently, 0 or an integer from 1 to 3;
r is an integer from 1 to 3;
or isomers, tautomers, carriers, prodrugs, and pharmaceutically acceptable salts thereof.

14. A compound according to claim 13 of formula (Ia) 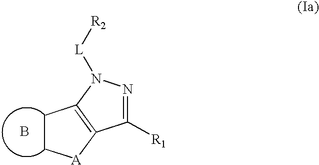
wherein B, R₁, L and R₂ are as defined in claim 13 and A is selected from the group consisting of —CH₂—, —CH₂—CH₂—, —CH═CH— and —CH₂—C(CH₃)₂—, the B ring being optionally further substituted as defined in claim 13.

15. A compound according to claim 13 of formula (Ib) 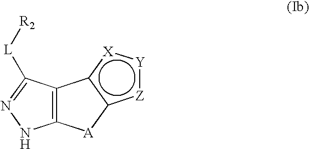
wherein X, Y, Z, L and R₂ are as defined in claim 13 and A is selected from the group consisting of —CH₂—, —CH₂—CH₂—, —CH═CH— and —CH₂—C(CH₃)₂—, the X, Y, Z ring being optionally further substituted as defined in claim 13.

16. A compound according to claim 13 of formula (Ic) 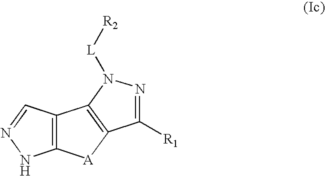
wherein R₁, L and R₂ are, each independently, as defined in claim 13, and A is selected from the group consisting of —CH₂—, —CH₂—CH₂—, —CH═CH— and —CH₂—C(CH₃)₂—.

17. A compound of formula (Ic) according to claim 16 wherein each L is independently selected from methylene or a single bond and each R₂ is independently selected from hydrido, phenyl or a 5 or 6 membered aromatic heterocycle having 1 or 2 heteroatoms selected among N, O and S.

18. A compound of formula (Ic) according to claim 17 wherein R₂, being optionally further substituted as defined in claim 13, is selected from the group consisting of hydrido phenyl, pyridyl, pyridazinyl or pyrimidinyl.

19. A compound of formula (Id) according to claim 13 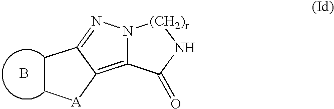
wherein r and B are as defined in claim 13, A is selected from the group consisting of —CH₂—, —CH₂—CH₂—, —CH═CH— and —CH₂—C(CH₃)₂—, and the B ring being optionally further substituted as defined in claim 13.

20. A compound of formula (Ie) or (If) according to claim 13 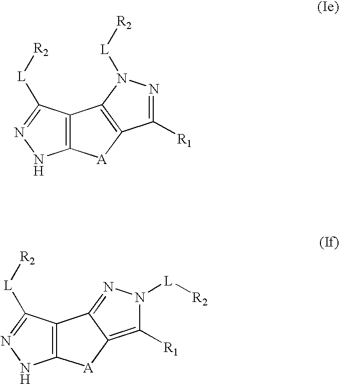
wherein L and R₂ are, each independently and the same or different in each occasion, as defined in claim 13; A is selected from the group consisting of —CH₂—CH₂—, —CH═CH— and —CH₂—C(CH₃)₂—; and R₁ is a group selected from NR′R″, CN, CO₂R′, COR′, CONR′R″, CONHOR′, CONHNH₂ and C(═NOH)NR′R″, wherein R′ and R″ are, the same or different, hydrido or lower alkyl.

21. A compound of formula (Ig) according to claim 13 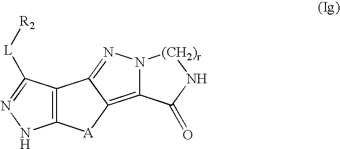
wherein L, R₂ and r are as defined in claim 13 and A is selected from the group consisting of —CH₂—CH₂—, —CH═CH— and —CH₂—C(CH₃)₂—.

22. A compound of formula (I) as defined in claim 13, optionally in the form of a pharmaceutically acceptable salt, selected from the group consisting of:
1. Ethyl 1-(4-methoxyphenyl)-1,4,5,6-tetrahydropyrazolo[3,4-e]indazole-3-carboxylate;
2. Ethyl 1-[4-(aminosulfonyl)phenyl]-1,4,5,6-tetrahydropyrazolo[3,4-e]indazole-3-carboxylate;
3. Ethyl 1-{4-[(methylamino)sulfonyl]phenyl}-1,4,5,6-tetrahydropyrazolo[3,4-e]indazole-3-carboxylate;
4. Ethyl 1-{4-[(butylamino)sulfonyl]phenyl}-1,4,5,6-tetrahydropyrazolo[3,4-e]indazole-3-carboxylate;
5. Ethyl 1-{4-[(dimethylamino)sulfonyl]phenyl}-1,4,5,6-tetrahydropyrazolo[3,4-e]indazole-3-carboxylate;
6. Ethyl 1-{4-[(diprop-2-ynylamino)sulfonyl]phenyl}-1,4,5,6-tetrahydropyrazolo[3,4-e]indazole-3-carboxylate;
7. Ethyl 1-[4-(anilinosulfonyl)phenyl]-1,4,5,6-tetrahydropyrazolo[3,4-e]indazole-3-carboxylate;
8. Ethyl 1-[4-(methylsulfonyl)phenyl]-1,4,5,6-tetrahydropyrazolo[3,4-e]indazole-3-carboxylate;
9. Ethyl 1-(4-{[(2-hydroxypropyl)amino]sulfonyl}phenyl)-1,4,5,6-tetrahydropyrazolo-[3,4-e]indazole-3-carboxylate;
10. Ethyl 1-[4-(aminocarbonyl)phenyl]-1,4,5,6-tetrahydropyrazolo[3,4-e]indazole-3-carboxylate;
11. Ethyl 1,4,5,6-tetrahydropyrazolo[3,4-e]indazole-3-carboxylate;
12. Ethyl 1-phenyl-1,4,5,6-tetrahydropyrazolo[3,4-e]indazole-3-carboxylate;
13. Ethyl 1-(4-fluorophenyl)-1,4,5,6-tetrahydropyrazolo[3,4-e]indazole-3-carboxylate;
14. Ethyl 1-(4-bromophenyl)-1,4,5,6-tetrahydropyrazolo[3,4-e]indazole-3-carboxylate;
15. Ethyl 1-(4-methylphenyl)-1,4,5,6-tetrahydropyrazolo[3,4-e]indazole-3-carboxylate;
16. Ethyl 1-(4-chlorophenyl) 1,4,5,6-tetrahydropyrazolo[3,4-e]indazole-3-carboxylate;
17. Ethyl 1-(4-cyanophenyl)-1,4,5,6-tetrahydropyrazolo[3,4-e]indazole-3-carboxylate;
18. Ethyl 1-(4-nitrophenyl)-1,4,5,6-tetrahydropyrazolo[3,4-e]indazole-3-carboxylate;
19. Ethyl 1-[4-(trifluoromethyl)phenyl]-1,4,5,6-tetrahydropyrazolo[3,4-e]indazole-3-carboxylate;
20. Ethyl 1-benzyl-1,4,5,6-tetrahydropyrazolo[3,4-e]indazole-3-carboxylate;
21. Ethyl 1-(3-hydroxybenzyl)-1,4,5,6-tetrahydropyrazolo[3,4-e]indazole-3-carboxylate;
22. Ethyl 1-pyridin-2-yl-1,4,5,6-tetrahydropyrazolo[3,4-e]indazole-3-carboxylate;
23. Ethyl 1-(6-chloropyridazin-3-yl)-1,4,5,6-tetrahydropyrazolo[3,4-e]indazole-3-carboxylate;
24. Ethyl 1-[4-(trifluoromethyl)pyrimidin-2-yl]-1,4,5,6-tetrahydropyrazolo[3,4-e]indazole-3-carboxylate;
25. Ethyl 1-(3-methylphenyl)-1,4,5,6-tetrahydropyrazolo[3,4-e]indazole-3-carboxylate;
26. Ethyl 1-(3-chlorophenyl)-1,4,5,6-tetrahydropyrazolo[3,4-e]indazole-3-carboxylate;
27. Ethyl 1-(3-fluorophenyl)-1,4,5,6-tetrahydropyrazolo[3,4-e]indazole-3-carboxylate;
28. Ethyl 4,4-dimethyl-1-(4-methylphenyl)-1,4,5,6-tetrahydropyrazolo[3,4-e]indazole-3-carboxylate;
29. Ethyl 1-pyridin-3-yl-1,4,5,6-tetrahydropyrazolo[3,4-e]indazole-3-carboxylate;
30. Ethyl 1-[4-(acetylamino)phenyl]-1,4,5,6-tetrahydropyrazolo[3,4-e]indazole-3-carboxylate;
31. Ethyl 1-{4-[(4-methylpiperazin-1-yl)sulfonyl]phenyl}-1,4,5,6-tetrahydropyrazolo[3,4-e]indazole-3-carboxylate;
32. 4-[3-(ethoxycarbonyl)-5,6-dihydropyrazolo[3,4-e]indazol-1(4H)-yl]benzoic acid;
33. Ethyl 1-[4-(trifluoromethoxy)phenyl]-1,4,5,6-tetrahydropyrazolo[3,4-e]indazole-3-carboxylate;
34. Ethyl 1-butyl-1,4,5,6-tetrahydropyrazolo[3,4-e]indazole-3-carboxylate;
35. Ethyl 1-(2,5-dimethylphenyl)-1,4,5,6-tetrahydropyrazolo[3,4-e]indazole-3-carboxylate;
36. Ethyl 1-{4-[amino(imino)methyl]phenyl}-1,4,5,6-tetrahydropyrazolo[3,4-e]indazole-3-carboxylate hydrochloride;
37. Ethyl 1-[4-(1H-imidazol-2-yl)phenyl]-1,4,5,6-tetrahydropyrazolo[3,4-e]indazole-3-carboxylate hydrochloride;
38. Ethyl 1-methyl-1,4,5,6-tetrahydropyrazolo[3,4-e]indazole-3-carboxylate;
39. Ethyl 8-anilino-1-methyl-1,4,5,6-tetrahydropyrazolo[3,4-e]indazole-3-carboxylate;
40. Ethyl 8-anilino-1-(2,2,2-trifluoroethyl)-1,4,5,6-tetrahydropyrazolo[3,4-e]indazole-3-carboxylate;
41. Ethyl 8-anilino-2-{2-[(tert-butoxycarbonyl)amino]ethyl}-2,4,5,6-tetrahydropyrazolo[3,4-e]indazole-3-carboxylate;
42. Ethyl 8-amino-1,4,5,6-tetrahydropyrazolo[3,4-e]indazole-3-carboxylate;
43. 1-(4-methoxyphenyl)-1,4,5,6-tetrahydro-pyrazolo[3,4-e]indazole-3-carboxamide;
44. 1-[4-(aminosulfonyl)phenyl]-1,4,5,6-tetrahydropyrazolo[3,4-e]indazole-3-carboxamide;
45. 1-{4-[(methylamino)sulfonyl]phenyl}-1,4,5,6-tetrahydropyrazolo[3,4-e]indazole-3-carboxamide;
46. 1-{4-[(butylamino)sulfonyl]phenyl}-1,4,5,6-tetrahydropyrazolo[3,4-e]indazole-3-carboxamide;
47. 1-{4-[(dimethylamino)sulfonyl]phenyl}-1,4,5,6-tetrahydropyrazolo[3,4-e]indazole-3-carboxamide;
48. 1-{4-[(diprop-2-ynylamino)sulfonyl]phenyl}-1,4,5,6-tetrahydropyrazolo[3,4-e]indazole-3-carboxamide;
49. 1-[4-(anilinosulfonyl)phenyl]-1,4,5,6-tetrahydropyrazolo[3,4-e]indazole-3-carboxamide;
50. 1-[4-(methylsulfonyl)phenyl]-1,4,5,6-tetrahydropyrazolo[3,4-e]indazole-3-carboxamide;
51. 1-[4-(anilinocarbonyl)phenyl]-1,4,5,6-tetrahydropyrazolo[3,4-e]indazole-3-carboxamide;
52. 1-(4-{[(2-hydroxypropyl)amino]sulfonyl}phenyl)-1,4,5,6-tetrahydropyrazolo[3,4-e]indazole-3-carboxamide;
53. 1,4,5,6-tetrahydropyrazolo[3,4-e]indazole-3-carboxamide;
54. 1-phenyl-1,4,5,6-tetrahydropyrazolo[3,4-e]indazole-3-carboxamide;
55. 1-(4-fluorophenyl)-1,4,5,6-tetrahydropyrazolo[3,4-e]indazole-3-carboxamide;
56. 1-(4-bromophenyl)-1,4,5,6-tetrahydropyrazolo[3,4-e]indazole-3-carboxamide;
57. 1-(4-nitrophenyl)-1,4,5,6-tetrahydropyrazolo[3,4-e]indazole-3-carboxamide;
58. 1-(4-methylphenyl)-1,4,5,6-tetrahydropyrazolo[3,4-e]indazole-3-carboxamide;
59. 1-(4-chlorophenyl)-1,4,5,6-tetrahydropyrazolo[3,4-e]indazole-3-carboxamide;
60. 1-(4-cyanophenyl)-1,4,5,6-tetrahydropyrazolo[3,4-e]indazole-3-carboxamide;
61. 1-[4-(trifluoromethyl)phenyl]-1,4,5,6-tetrahydropyrazolo[3,4-e]indazole-3-carboxamide;
62. 1-benzyl-1,4,5,6-tetrahydropyrazolo[3,4-e]indazole-3-carboxamide;
63. 1-(3-hydroxybenzyl)-1,4,5,6-tetrahydropyrazolo[3,4-e]indazole-3-carboxamide;
64. 1-pyridin-2-yl-1,4,5,6-tetrahydropyrazolo[3,4-e]indazole-3-carboxamide;
65. 1-(3-methylphenyl)-1,4,5,6-tetrahydropyrazolo[3,4-e]indazole-3-carboxamide;
66. 1-(3-chlorophenyl)-1,4,5,6-tetrahydropyrazolo[3,4-e]indazole-3-carboxamide;
67. 1-(3-fluorophenyl)-1,4,5,6-tetrahydropyrazolo[3,4-e]indazole-3-carboxamide;
68. 1-(6-chloropyridazin-3-yl)-1,4,5,6-tetrahydropyrazolo[3,4-e]indazole-3-carboxamide;
69. 4,4-dimethyl-1-(4-methylphenyl)-1,4,5,6-tetrahydropyrazolo[3,4-e]indazole-3-carboxamide;
70. 1-pyridin-3-yl-1,4,5,6-tetrahydropyrazolo[3,4-e]indazole-3-carboxamide;
71. 1-[4-(acetylamino)phenyl]-1,4,5,6-tetrahydropyrazolo[3,4-e]indazole-3-carboxamide;
72. 1-(4-aminophenyl)-1,4,5,6-tetrahydropyrazolo[3,4-e]indazole-3-carboxamide;
73. 1-{4-[(4-methylpiperazin-1-yl)sulfonyl]phenyl}-1,4,5,6-tetrahydropyrazolo[3,4-e]indazole-3-carboxamide;
74. 4-[3-(aminocarbonyl)-5,6-dihydropyrazolo[3,4-e]indazol-1(4H)-yl]benzoic acid;
75. 1-(4-morpholin-4-ylphenyl)-1,4,5,6-tetrahydropyrazolo[3,4-e]indazole-3-carboxamide;
76. 1-[4-(trifluoromethoxy)phenyl]-1,4,5,6-tetrahydropyrazolo[3,4-e]indazole-3-carboxamide;
77. 1-butyl-1,4,5,6-tetrahydropyrazolo[3,4-e]indazole-3-carboxamide;
78. 1-(2-hydroxyethyl)-1,4,5,6-tetrahydropyrazolo[3,4-e]indazole-3-carboxamide;
79. 1-(2,5-dimethylphenyl)-1,4,5,6-tetrahydropyrazolo[3,4-e]indazole-3-carboxamide;
80. 1-(2,2,2-trifluoroethyl)-1,4,5,6-tetrahydropyrazolo[3,4-e]indazole-3-carboxamide;
81. 1-(2-amino-2-oxoethyl)-1,4,5,6-tetrahydropyrazolo[3,4-e]indazole-3-carboxamide;
82. 1-[4-(1H-imidazol-2-yl)phenyl]-1,4,5,6-tetrahydropyrazolo[3,4-e]indazole-3-carboxamide;
83. 4,4-dimethyl-1-(2,2,2-trifluoroethyl)-1,4,5,6-tetrahydropyrazolo[3,4-e]indazole-3-carboxamide;
84. 1-methyl-1,4,5,6-tetrahydropyrazolo[3,4-e]indazole-3-carboxamide;
85. 2-(2-hydroxyethyl)-2,4,5,6-tetrahydropyrazolo[3,4-e]indazole-3-carboxamide;
86. 8-Anilino-1-methyl-1,4,5,6-tetrahydropyrazolo[3,4-e]indazole-3-carboxamide;
87. 8-Anilino-1-(2,2,2-trifluoroethyl)-1,4,5,6-tetrahydropyrazolo[3,4-e]indazole-3-carboxamide;
88. 8-amino-1,4,5,6-tetrahydropyrazolo[3,4-e]indazole-3-carboxamide;
89. 1-[4-methoxyphenyl]-1,6-dihydropyrazolo[3,4-e]indazole-3-carboxamide;
90. 1-[4-(aminosulfonyl)phenyl]-1,6-dihydropyrazolo[3,4-e]indazole-3-carboxamide;
91.1-{4-[(methylamino)sulfonyl]phenyl}-1,6-dihydropyrazolo[3,4-e]indazole-3-carboxamide;
92. 1-{4-[(butylamino)sulfonyl]phenyl}-1,6-dihydropyrazolo[3,4-e]indazole-3-carboxamide;
93. 1-{4-[(dimethylamino)sulfonyl]phenyl}-1,6-dihydropyrazolo[3,4-e]indazole-3-carboxamide;
94. 1-{4-[(diprop-2-ynylamino)sulfonyl]phenyl}-1,6-dihydropyrazolo[3,4-e]indazole-3-carboxamide;
95. 1-[4-(anilinosulfonyl)phenyl]-1,6-dihydropyrazolo[3,4-e]indazole-3-carboxamide;
96. 1-(4-{[(2-hydroxypropyl)amino]sulfonyl}phenyl)-1,6-dihydropyrazolo[3,4-e]indazole-3-carboxamide;
97. 1-[4-(methylsulfonyl)phenyl]-1,6-dihydropyrazolo[3,4-e]indazole-3-carboxamide;
98. 1,6-dihydropyrazolo[3,4-e]indazole-3-carboxamide;
99. 1-phenyl-1,6-dihydropyrazolo[3,4-e]indazole-3-carboxamide;
100. 1-(4-fluorophenyl)-1,6-dihydropyrazolo[3,4-e]indazole-3-carboxamide;
101. 1-(4-methylphenyl)-1,6-dihydropyrazolo[3,4-e]indazole-3-carboxamide;
102. 1-(4-cyanophenyl)-1,6-dihydropyrazolo[3,4-e]indazole-3-carboxamide;
103. 1-[4-(trifluoromethyl)phenyl]-1,6-dihydropyrazolo[3,4-e]indazole-3-carboxamide;
104. 1-(4-chlorophenyl)-1,6-dihydropyrazolo[3,4-e]indazole-3-carboxamide;
105. 1-(4-bromophenyl)-1,6-dihydropyrazolo[3,4-e]indazole-3-carboxamide;
106. 1-(4-nitrophenyl)-1,6-dihydropyrazolo[3,4-e]indazole-3-carboxamide;
107. 1-benzyl-1,6-dihydropyrazolo[3,4-e]indazole-3-carboxamide;
108. 1-(3-hydroxybenzyl)-1,6-dihydropyrazolo[3,4-e]indazole-3-carboxamide;
109. 1-pyridin-2-yl-1,6-dihydropyrazolo[3,4-e]indazole-3-carboxamide;
110. 1-(3-chlorophenyl)-1,6-dihydropyrazolo[3,4-e]indazole-3-carboxamide;
111. 1-(3-methylphenyl)-1,6-dihydropyrazolo[3,4-e]indazole-3-carboxamide;
112. 1-(3-fluorophenyl)-1,6-dihydropyrazolo[3,4-e]indazole-3-carboxamide;
113. 1-(6-chloropyridazin-3-yl)-1,6-dihydropyrazolo[3,4-e]indazole-3-carboxamide;
114. 1-(4-methoxyphenyl)-1,6-dihydropyrazolo[3,4-e]indazole-3-carboxylic acid;
115. Ethyl 1-phenyl-1,6-dihydropyrazolo[3,4-e]indazole-3-carboxylate;
116. Ethyl 1-(4-methoxyphenyl)-1,6-dihydropyrazolo[3,4-e]indazole-3-carboxylate;
117. N-methyl-1-[4-(aminosulfonyl)phenyl]-1,6-dihydropyrazolo[3,4-e]indazole-3-carboxamide;
118. N-methyl-1-{4-[(butylamino)sulfonyl]phenyl}-1,6-dihydropyrazolo[3,4-e]indazole-3-carboxamide;
119. N-methyl-1-{4-[(dimethylamino)sulfonyl]phenyl}-1,6-dihydropyrazolo[3,4-e]indazole-3-carboxamide;
120. N-methyl-1-[4-(methylsulfonyl)phenyl]-1,6-dihydropyrazolo[3,4-e]indazole-3-carboxamide;
121. N-(allyloxy)-1-{4-[(butylamino)sulfonyl]phenyl}-1,6-dihydropyrazolo[3,4-e]indazole-3-carboxamide;
122. 7,8,9,10-tetrahydro[1,4]diazepino[1,2-b]pyrazolo[3,4-g]indazol-6(3H)-one;
123. 1-pyridin-3-yl-1,6-dihydropyrazolo[3,4-e]indazole-3-carboxamide;
124. 1-[4-(acetylamino)phenyl]-1,6-dihydropyrazolo[3,4-e]indazole-3-carboxamide,
125. 4-[3-(aminocarbonyl)pyrazolo[3,4-e]indazol-1 (6H)-yl]benzoic acid;
126. 1-{4-[(4-methylpiperazin-1-yl)sulfonyl]phenyl}-1,6-dihydropyrazolo[3,4-e]indazole-3-carboxamide;
127. 1-[4-(trifluoromethoxy)phenyl]-1,6-dihydropyrazolo[3,4-e]indazole-3-carboxamide;
128. 4-[3-(ethoxycarbonyl)pyrazolo[3,4-e]indazol-1 (6H)-yl]benzoic acid;
129. 1-(4-morpholin-4-ylphenyl)-1,6-dihydropyrazolo[3,4-e]indazole-3-carboxamide;
130. 1-(2-hydroxyethyl)-1,6-dihydropyrazolo[3,4-e]indazole-3-carboxamide;
131. 1-(2,5-dimethylphenyl)-1,6-dihydropyrazolo[3,4-e]indazole-3-carboxamide;
132. 1-(2-aminoethyl)-1,6-dihydropyrazolo[3,4-e]indazole-3-carboxamide hydrochloride;
133. 1-(2,2,2-trifluoroethyl)-1,6-dihydropyrazolo[3,4-e]indazole-3-carboxamide;
134. 1-[4-(1H-imidazol-2-yl)phenyl]-1,6-dihydropyrazolo[3,4-e]indazole-3-carboxamide;
135. 1-methyl-1,6-dihydropyrazolo[3,4-e]indazole-3-carboxamide;
136. 8,9-dihydro-3H-pyrazino[1,2-b]pyrazolo[3,4-g]indazol-6(7H)-one;
137. 2-(2-aminoethyl)-2,6-dihydropyrazolo[3,4-e]indazole-3-carboxamide hydrochloride;
138. 2-(2-hydroxyethyl)-2,6-dihydropyrazolo[3,4-e]indazole-3-carboxamide;
139. 2-methyl-2,6-dihydropyrazolo[3,4-e]indazole-3-carboxamide;
140. 1-anilino-8,9-dihydro-3H-pyrazino[1,2-b]pyrazolo[3,4-g]indazol-6(7H)-one;
141. 1-(4-methoxy-phenyl)-1,6-dihydropyrazolo[3,4-e]indazol-3-amine;
142. 1-[1-(4-methylphenyl)-1,6-dihydropyrazolo[3,4-e]indazol-3-yl]ethanone;
143. 1-(4-methoxyphenyl)-1,6-dihydropyrazolo[3,4-e]indazole-3-carbonitrile;
144. 1-(4-methoxyphenyl)-1,6-dihydropyrazolo[3,4-e]indazole-3-carbohydrazide;
145. 1-(4-methoxyphenyl)-1,4,5,6-tetrahydropyrazolo[3,4-e]indazole-3-carbohydrazide;
146. N′-hydroxy-1-(4-methoxyphenyl)-1,6-dihydropyrazolo[3,4-e]indazole-3-carboximidamide;
147. 1-(4-methoxyphenyl)-1,6-dihydropyrazolo[3,4-e]indazole-3-carboxylic acid;
148. 1-(4-bromophenyl)-1,4,5,6-tetrahydropyrazolo[3,4-e]indazole-3-carboxylic acid;
149. 1-{4-[(butylamino)sulfonyl]phenyl}-1,4,5,6-tetrahydropyrazolo[3,4-e]indazole-3-carboxylic acid;
150. 4,4-dimethyl-1-(4-methylphenyl)-1,4,5,6-tetrahydropyrazolo[3,4-e]indazole-3-carboxylic acid;
151. N-hydroxy-1-(4-methoxyphenyl)-1,6-dihydropyrazolo[3,4-e]indazole-3-carboxamide;
152. N-(allyloxy)-1-{4-[(butylamino)sulfonyl]phenyl}-1,4,5,6-tetrahydropyrazolo[3,4-e]indazole-3-carboxamide;
153. N-(allyloxy)-1-(4-methoxyphenyl)-1,4,5,6-tetrahydropyrazolo[3,4-e]indazole-3-carboxamide;
154. N-methyl-1-[4-(methylsulfonyl)phenyl]-1,4,5,6-tetrahydropyrazolo[3,4-e]indazole-3-carboxamide;
155. 1-[4-(aminosulfonyl)phenyl]-N-methyl-1,4,5,6-tetrahydropyrazolo[3,4-e]indazole-3-carboxamide;
156. 1-{4-[(butylamino)sulfonyl]phenyl}-N-methyl-1,4,5,6-tetrahydropyrazolo[3,4-e]indazole-3-carboxamide;
157. 1-{4-[(dimethylamino)sulfonyl]phenyl}-N-methyl-1,4,5,6-tetrahydropyrazolo[3,4-e]indazole-3-carboxamide;
158. Ethyl 2-(3-aminopropyl)-2,4,5,6-tetrahydropyrazolo[3,4-e]indazole-3-carboxylate hydrochloride;
159. 4,5,7,8,9,10-hexahydro[1,4]diazepino[1,2-b]pyrazolo[3,4-g]indazol-6(3H)-one;
160. 5,5-dimethyl-4,5,7,8,9,10-hexahydro[1,4]diazepino[1,2-b]pyrazolo[3,4-g]indazol-6(3H)-one;
161. 5,5-dimethyl-4,5,8,9-tetrahydro-3H-pyrazino[1,2-b]pyrazolo[3,4-g]indazol-6(7H)-one;
162. 4,5,8,9-tetrahydro-3H-pyrazino[1,2-b]pyrazolo[3,4-g]indazol-6(7H)-one;
163. 1-anilino-4,5,8,9-tetrahydro-3H-pyrazino[1,2-b]pyrazolo[3,4-g]indazol-6(7H)-one.

23. A process for preparing a compound of formula (Ic) as defined in claim 16 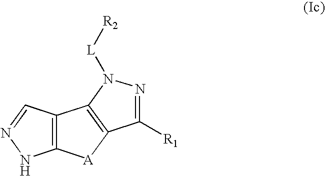
wherein L and R₂ are as defined in claim 16, R₁ is a group —COOEt or —CONH₂, and A is selected from the group consisting of —CH₂—, —CH₂—CH₂—, —CH═CH— and —CH₂—C(CH₃)₂—, which process comprises:
a) reacting the compound (10) with hydrazine dihydrochloride, so as to obtain the compound (11) 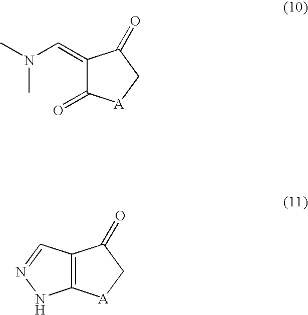
wherein A is as above defined, other than —CH═CH—;
b) reacting the compound (11) with trityl chloride, so as to obtain the compound (12) 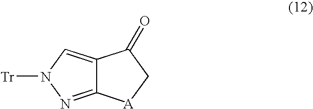
wherein Tr stands for trityl, and condensing it with oxalyl chloride so as to obtain the compound (13) 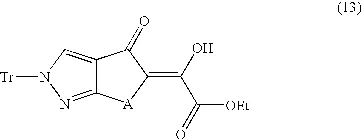
c) reacting the compound (13) with a substituted hydrazine (8) 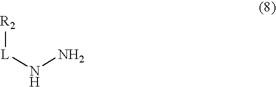
wherein L and R₂ are as defined in claim 16; so as to obtain a compound of formula (Ic) wherein R₁ is a group —COOEt and A is as above defined except —CH═CH—; and, optionally
d) reacting this latter with ammonium hydroxide so as to obtain the corresponding derivative of formula (Ic) wherein R₁ is —CONH₂; and, optionally
e) reacting the compound of formula (Ic) wherein A is —CH₂—CH₂—, as obtained in steps c) or d), with a suitable oxidizing agent so as to obtain the corresponding derivative of formula (Ic) wherein A is —CH═CH—.

24. The process of claim 23 wherein, in step e), the oxidizing agent is 2,3-dichloro-5,6-dicyano-1,4-benzoquinone.

25. The compound of formula (11) 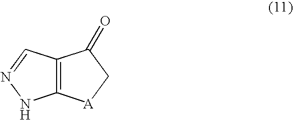
wherein A is selected from —CH₂— or —CH₂—CH₂—.

26. The compounds of formula (12) and (13) 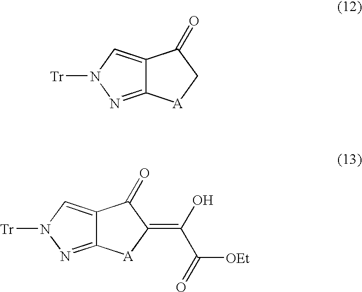
wherein Tr is trityl and A is selected from —CH₂—, —CH₂—CH₂— and —CH₂—C(CH₃)₂—.

27. A pharmaceutical composition comprising an effective amount of a compound of formula (I) as defined in claim 13 and, at least, one pharmaceutically acceptable excipient, carrier or diluent.

28. A pharmaceutical composition according to claim 27 further comprising one or more chemotherapeutic agents, as a combined preparation for simultaneous, separate or sequential use in anticancer therapy.

29. A product or kit comprising a compound of claim 13 or a pharmaceutical composition thereof as defined in claim 27, and one or more chemotherapeutic agents, as a combined preparation for simultaneous, separate or sequential use in anticancer therapy.

30. A compound of formula (I) or a pharmaceutically acceptable salt thereof, as defined in claim 13, for use as a medicament.

31. Use of a compound of formula (I) or a pharmaceutically acceptable salt thereof, as defined in claim 13, in the manufacture of a medicament for treating diseases caused by and/or associated with an altered protein kinase activity.

32. Use according to claim 31 for treating tumors.
